(19) United States
(12) Patent Application Publication
(10) Pub. No.: US 2014/0207159 A1
(43) Pub. Date: Jul. 24, 2014

(54) DEVICES AND METHODS FOR DELIVERING AN ANCHORED DEVICE

(71) Applicant: ValenTx, Inc., Carpinteria, CA (US)

(72) Inventors: Sean MILLER, Santa Barbara, CA (US); Cole CHEN, Ventura, CA (US); Terry DAHL, Santa Barbara, CA (US); Johann NEISZ, Minneapolis, MN (US); Jesse N. NELSON, Carpinteria, CA (US); Allen PUTNAM, Minneapolis, MN (US); Christopher THIERFELDER, Santa Barbara, CA (US); Josiah VERKAIK, Rocklin, CA (US); James WRIGHT, Santa Barbara, CA (US)

(21) Appl. No.: 14/164,112

(22) Filed: Jan. 24, 2014

Related U.S. Application Data
(60) Provisional application No. 61/756,366, filed on Jan. 24, 2013.

(51) Int. Cl. A61B 17/04

(52) U.S. Cl. CPC A61B 17/0401
USPC 606/151

(57) ABSTRACT

One or more tissue anchors may be delivered first without being coupled to an anchored device. An anchored device may be delivered second, and then coupled to the tissue anchors. This may enhance placement accuracy of tissue anchors, as well as facilitate delivery of tissue anchors. The tissue anchors include anchor couplings configured to be coupled to one or more device couplings of the anchored device.

### CROSS-REFERENCE TO RELATED APPLICATION

This application claims the benefit of U.S. provisional patent application Ser. No. 61/756,366, filed Jan. 24, 2013, which is hereby incorporated by reference in its entirety.

### BACKGROUND

Tissue anchors such as those described in U.S. Pat. No. 8,070,743, and U.S. patent application Ser. Nos. 12/137,473, 13/485,887, and 13/896,838 may be used to anchor devices within a bodily space. For example, tissue anchors may be used to anchor a gastrointestinal bypass device within an esophageal lumen.

The function and longevity of an anchored device may be affected by the placement of the tissue anchors. For example, tissue anchors that are uniformly spaced or substantially coplanar may improve the performance and increase the useful life of an anchored device.

An anchored device may need to be coupled to the tissue anchors before the tissue anchors are delivered through the tissue wall. However, an anchored device coupled to the tissue anchors may interfere with and complicate placement and delivery of the tissue anchors.

What is needed are devices and methods to enhance placement accuracy of tissue anchors for anchoring of an anchored device. What is also needed are devices and methods to facilitate delivery of tissue anchors for anchoring of an anchored device.

### SUMMARY

A method of treating a patient for a condition is described. The condition may include diabetes, heart disease, obesity, obesity-related conditions, and/or other chronic conditions. The method comprises providing a tissue anchor including a tension element, an anchor coupling coupled to a proximal portion of the tension element, and a distal retention element coupled to a distal portion of the tension element. The anchor coupling may be configured to be positioned within a bodily space. The distal retention element may be configured to be deployed on a distal side of a tissue wall defining the bodily space. The method also comprises selecting a placement in the tissue wall for the tissue anchor, delivering the distal retention element through the placement in the tissue wall, deploying the distal retention element on the distal side of the tissue wall, and positioning the anchor coupling within the bodily space. The method also comprises providing an anchored device configured to be positioned within the bodily space. The anchored device includes a device coupling configured to be coupled to the anchor coupling. The method also comprises delivering the anchored device into the bodily space, and coupling the device coupling to the anchor coupling to anchor the anchored device in the bodily space.

### BRIEF DESCRIPTION OF THE DRAWINGS

FIG. 1A shows one embodiment of a tissue anchor 5300 including a loop 6010. FIG. 1B shows one embodiment of an anchored device 5100 including one or more clips 6310. FIGS. 1C-1D show enlarged views of two embodiments of clip 6310. FIG. 1E shows one embodiment of a method for attaching anchored device 5100 within a bodily space S.

FIG. 2A shows one embodiment of a tissue anchor 5300 including a loop 6010. FIG. 2B shows one embodiment of an anchored device 5100 including one or more barbs 6320. FIGS. 2C-2D show enlarged views of two embodiments of barb 6320. FIG. 2E shows one embodiment of a method for attaching anchored device 5100 within a bodily space S.

FIG. 3A shows one embodiment of a tissue anchor 5300 including a barb 6020. FIGS. 3B-3C show enlarged views of two embodiments of barb 6020.

FIG. 3D shows one embodiment of an anchored device 5100 including one or more loops 6330. FIG. 3E shows one embodiment of a method for attaching anchored device 5100 within a bodily space S.

FIG. 4A shows one embodiment of a tissue anchor 5300 including a loop 6010. FIG. 4B shows one embodiment of an anchored device 5100 including one or more hitches 6340. FIGS. 4C-4D show enlarged views of two embodiments of hitch 6340. FIG. 4E shows one embodiment of a method for attaching anchored device 5100 within a bodily space S.

FIG. 5A shows one embodiment of a tissue anchor 5300 including a loop 6010. FIG. 5B shows one embodiment of an anchored device 5100 including one or more struts 6350. FIGS. 5C-5D show enlarged views of two embodiments of strut 6350. FIG. 5E shows one embodiment of a method for attaching anchored device 5100 within a bodily space S.

FIG. 6A shows one embodiment of a tissue anchor 5300 including a loop 6010. FIG. 6B shows one embodiment of an anchored device 5100 including one or more fins 6370. FIG. 6C shows an enlarged view of one embodiment of fin 6370. FIG. 6D shows one embodiment of a method for attaching anchored device 5100 within a bodily space S.

FIG. 7A shows one embodiment of a tissue anchor 5300 including a loop 6010. FIG. 7B shows one embodiment of an anchored device 5100 including one or more struts 6380. FIG. 7C shows an enlarged view of one embodiment of strut 6380. FIG. 7D shows one embodiment of a method for attaching anchored device 5100 within a bodily space S.

FIG. 8A shows one embodiment of a tissue anchor 5300 including a loop 6010. FIG. 8B shows one embodiment of an anchored device 5100 including a loop 6330 and a loop coupling 6400. FIGS. 8C-8D show enlarged views of two embodiments of loop coupling 6400. FIG. 8E shows one embodiment of a method for attaching anchored device 5100 within a bodily space S.

FIG. 9A shows one embodiment of a tissue anchor 5300 including a hook 6030. FIG. 9B shows an enlarged view of one embodiment of hook 6030. FIG. 9C shows one embodiment of an anchored device 5100 including a halo 6410. FIG. 9D shows one embodiment of a method for attaching anchored device 5100 within a bodily space S.

FIG. 10A shows one embodiment of a tissue anchor 5300 including a ball 6040 and a socket 6041. FIG. 10B shows an enlarged view of one embodiment of ball 6040 and socket 6141. FIG. 10C shows one embodiment of an anchored device 5100 including a halo 6410. FIG. 10D shows one embodiment of a method for attaching anchored device 5100 within a bodily space S.

FIG. 11A shows one embodiment of a tissue anchor 5300 including a fork 6050. FIGS. 11B-11C show enlarged views of one embodiment of fork 6050. FIG. 11D shows one embodiment of an anchored device 5100 including a halo 6410. FIG. 11E shows one embodiment of a method for attaching anchored device 5100 within a bodily space S.

FIG. 12A shows one embodiment of a tissue anchor 5300 including a clip 6060. FIG. 12B shows an enlarged view of one embodiment of clip 6060. FIG. 12C shows one embodiment of an anchored device 5100 including a halo 6420. FIG. 12D shows an enlarged view of halo 6420. FIG. 12E shows one embodiment of a method for attaching anchored device 5100 within a bodily space S.

FIG. 13A shows one embodiment of a tissue anchor 5300 including a loop 6010 and a halo 6070. FIG. 13B shows one embodiment of an anchored device 5100 including one or more clips 6310. FIG. 13C shows one embodiment of a method for attaching anchored device 5100 within a bodily space S.

FIG. 14A shows one embodiment of a tissue anchor 5300 including a cinching loop 6080. FIG. 14B shows one embodiment of an anchored device 5100 including one or more knobs 6430. FIG. 14C shows an enlarged view of one embodiment of knob 6430. FIG. 14D shows one embodiment of a method for attaching anchored device 5100 within a bodily space S.

FIG. 15A shows one embodiment of a tissue anchor 5300 including a button 6090. FIG. 15B shows one embodiment of an anchored device 5100 including one or more cinching loops 6440. FIG. 15C shows one embodiment of a method for attaching anchored device 5100 within a bodily space S.

FIG. 16A shows one embodiment of a tissue anchor 5300 including a button 6090. FIG. 16B shows one embodiment of an anchored device 5100 including one or more cinching loops 6450. FIG. 16C shows an enlarged view of one embodiment of cinching loop 6450. FIG. 16D shows one embodiment of a method for attaching anchored device 5100 within a bodily space S.

FIG. 17A shows one embodiment of a tissue anchor 5300 including a T-tag 6100. FIG. 17B shows an enlarged view of one embodiment of T-tag 6100. FIG. 17C shows one embodiment of an anchored device 5100 including one or more cinching loops 6440. FIG. 17D shows one embodiment of a method for attaching anchored device 5100 within a bodily space S.

FIG. 18A shows one embodiment of a tissue anchor 5300 including a button 6090. FIG. 18B shows one embodiment of an anchored device 5100 including one or more cinching loops 6470. FIGS. 18C-18D show enlarged views of one embodiment of cinching loop 6470 in an uncinched and cinched configurations. FIG. 18E-18H shows various embodiments of cinching loop 6470. FIG. 18I shows one embodiment of a method for attaching anchored device 5100 within a bodily space S.

FIG. 19A shows one embodiment of a tissue anchor 5300 including a button 6090. FIG. 19B shows one embodiment of an anchored device 5100 including one or more struts 6500. FIGS. 19C-19D show enlarged views of one embodiment of strut 6500 in an uncinched and cinched configurations. FIG. 19E shows one embodiment of a method for attaching anchored device 5100 within a bodily space S.

FIG. 20A shows one embodiment of a tissue anchor 5300 including a button 6090. FIG. 20B shows one embodiment of an anchored device 5100 including one or more struts 6520. FIGS. 20C-20H show enlarged views of various embodiments of strut 6520. FIG. 20I shows one embodiment of a method for attaching anchored device 5100 within a bodily space S.

FIG. 21A shows one embodiment of a tissue anchor 5300 including one or more balls 6110. FIG. 21B shows one embodiment of an anchored device 5100 including one or more struts 6540. FIGS. 21C-21D shows enlarged views of two embodiments of strut 6540. FIG. 21E shows one embodiment of a method for attaching anchored device 5100 within a bodily space S.

FIG. 22A shows one embodiment of a tissue anchor 5300 including a knob 6120. FIG. 22B shows one embodiment of an anchored device 5100 including one or more struts 6560. FIG. 22C shows an enlarged view of one embodiment of strut 6560. FIG. 22D shows one embodiment of a method for attaching anchored device 5100 within a bodily space S.

FIG. 23A shows one embodiment of a tissue anchor 5300 including an insert 6130. FIGS. 23B-23C show enlarged views of various embodiments of insert 6130. FIG. 23D shows one embodiment of an anchored device 5100 including an attachment ring 6580. FIG. 23E shows one embodiment of a method for attaching anchored device 5100 within a bodily space S.

FIG. 24A shows one embodiment of a tissue anchor 5300 including an insert 6130. FIG. 24B shows one embodiment of an anchored device 5100 including one or more struts 6590. FIGS. 24C-24D show enlarged views of two embodiments of strut 6590. FIG. 24E shows one embodiment of a method for attaching anchored device 5100 within a bodily space S.

FIG. 25A shows one embodiment of a tissue anchor 5300 including a pop cap 6140. FIGS. 25B-25C show enlarged views of one embodiment of pop cap 6140. FIG. 25D shows one embodiment of an anchored device 5100 including one or more knobs 6430. FIG. 25E shows one embodiment of a method for attaching anchored device 5100 within a bodily space S.

FIG. 26A shows one embodiment of a tissue anchor 5300 including a pincer 6150. FIG. 26B shows an enlarged view of one embodiment of pincer 6150. FIG. 26C shows one embodiment of an anchored device 5100 including one or more blocks 6610. FIG. 26D shows an enlarged view of one embodiment of block 6610. FIG. 26E shows one embodiment of a method for attaching anchored device 5100 within a bodily space S.

FIG. 27A shows one embodiment of a tissue anchor 5300 including one or more balls 6110. FIG. 27B shows one embodiment of an anchored device 5100 including one or more struts 6620. FIGS. 27C-27D show enlarged views of one embodiment of strut 6620. FIG. 27E shows one embodiment of a method for attaching anchored device 5100 within a bodily space S.

FIG. 28A shows one embodiment of a tissue anchor 5300 including one or more balls 6110. FIG. 28B shows one embodiment of an anchored device 5100 including one or more struts 6640. FIGS. 28C-28D show enlarged views of one embodiment of strut 6640. FIG. 28E shows one embodiment of a method for attaching anchored device 5100 within a bodily space S.

FIG. 29A shows one embodiment of a tissue anchor 5300 including one or more balls 6110. FIG. 29B shows one embodiment of an anchored device 5100 including one or more struts 6660. FIG. 29C shows an enlarged view of one embodiment of strut 6660. FIG. 29D shows one embodiment of a method for attaching anchored device 5100 within a bodily space S.

FIG. 30A shows one embodiment of a tissue anchor 5300 including one or more balls 6110. FIG. 30B shows one embodiment of an anchored device 5100 including one or more struts 6680. FIGS. 30C-30D show enlarged views of one embodiment of strut 6680. FIG. 30E shows one embodiment of a method for attaching anchored device 5100 within a bodily space S.

FIG. 31A shows one embodiment of a tissue anchor 5300 including one or more balls 6110. FIG. 31B shows one embodiment of an anchored device 5100 including one or more struts 6700. FIG. 31C shows an enlarged view of one embodiment of strut 6700. FIG. 31D shows one embodiment of a method for attaching anchored device 5100 within a bodily space S.

FIG. 32A shows one embodiment of a tissue anchor 5300 including one or more balls 6110. FIG. 32B shows one embodiment of an anchored device 5100 including one or more struts 6720. FIG. 32C shows an enlarged view of one embodiment of strut 6720. FIG. 32D shows one embodiment of a method for attaching anchored device 5100 within a bodily space S.

FIG. 33A shows one embodiment of a tissue anchor 5300. FIG. 33B shows one embodiment of an anchored device 5100 including one or more struts 6740. FIG. 33C shows an enlarged view of one embodiment of strut 6740. FIG. 33D shows one embodiment of a method for attaching anchored device 5100 within a bodily space S.

FIG. 34A shows one embodiment of a tissue anchor 5300 including one or more balls 6110. FIG. 34B shows one embodiment of an anchored device 5100 including one or more struts 6760. FIGS. 34C-34D show enlarged views of one embodiment of strut 6760. FIG. 34E shows one embodiment of a method for attaching anchored device 5100 within a bodily space S.

FIG. 35A shows one embodiment of a tissue anchor 5300 including an expanding element 6160. FIG. 35B shows one embodiment of an anchored device 5100 including one or more holes 6780. FIG. 35C shows one embodiment of a method for attaching anchored device 5100 within a bodily space S.

FIG. 36A shows one embodiment of a tissue anchor 5300 including a magnet 6170. FIG. 36B shows one embodiment of an anchored device 5100 including one or more magnets 6790. FIG. 36C shows one embodiment of a method for attaching anchored device 5100 within a bodily space S.

FIG. 37A shows one embodiment of a tissue anchor 5300 including a button 6090. FIG. 37B shows one embodiment of an anchored device 5100 including one or more struts 6800. FIG. 37C shows an enlarged view of one embodiment of strut 6800. FIG. 37D shows one embodiment of a method for attaching anchored device 5100 within a bodily space S.

FIG. 38A shows one embodiment of a tissue anchor 5300 including a suture lock 6180. FIG. 38B shows an enlarged view of one embodiment of suture lock 6180. FIG. 38C shows one embodiment of an anchored device 5100 including one or more holes 6780. FIG. 38D shows one embodiment of a method for attaching anchored device 5100 within a bodily space S.

FIG. 39A shows a cross-sectional view of one embodiment of a tissue anchor 5300 including a ball 6200. FIG. 39B shows a cross-sectional view of one embodiment of a socket 6820 of an anchored device 5100. FIG. 39C shows one embodiment of a method for attaching anchored device 5100 within a bodily space S.

FIG. 40A shows one embodiment of a tissue anchor 5300 including a pin 6210. FIG. 40B shows one embodiment of a clip 6830 of an anchored device 5100. FIG. 40C shows one embodiment of a method for attaching anchored device 5100 within a bodily space S.

FIG. 41A shows one embodiment of a tube 6220 of a tissue anchor 5300. FIG. 41B shows one embodiment of a pin 6840 of an anchored device 5100. FIG. 41C shows one embodiment of a method for attaching anchored device 5100 within a bodily space S.

FIG. 42A shows one embodiment of a clip 6230 of a tissue anchor 5300. FIG. 42B shows one embodiment of a pin 6850 of an anchored device 5100. FIG. 42C shows one embodiment of a method for attaching anchored device 5100 within a bodily space S.

FIG. 43A shows one embodiment of a clip 6240 of a tissue anchor 5300. FIG. 43B shows one embodiment of a pin 6860 of an anchored device 5100. FIG. 43C shows one embodiment of a method for attaching anchored device 5100 within a bodily space S.

FIG. 44A shows one embodiment of a tissue anchor 5300 including a knob 6250. FIG. 44B shows one embodiment of a clip 6870 of an anchored device 5100. FIG. 44C shows one embodiment of a method for attaching anchored device 5100 within a bodily space S.

FIG. 45 shows one embodiment of a tissue anchor 5300 including a barb 6260. FIG. 45 also shows one embodiment of an anchored device 5100 including one or more holes 6880. FIG. 45 shows one embodiment of a method for attaching anchored device 5100 within a bodily space S.

FIG. 46A shows one embodiment of a ball 6270 of a tissue anchor 5300. FIG. 46A also shows one embodiment of a socket 6890 of an anchored device 5100. FIGS. 46B-46D show one embodiment of a method for coupling ball 6270 to socket 6890.

FIGS. 47A-47B show side and front views of one embodiment of a tissue anchor 5300 including a folding member 6280. FIGS. 47C-47D show side and front views of another embodiment of a tissue anchor 5300 including a folding member 6280.

### DESCRIPTION

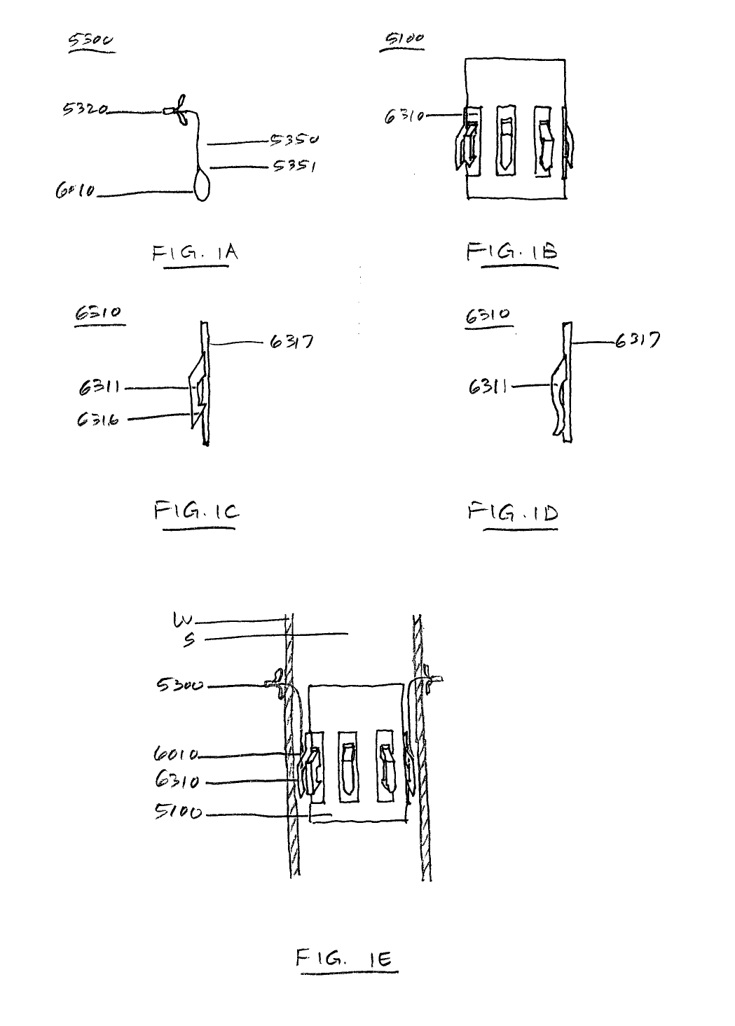

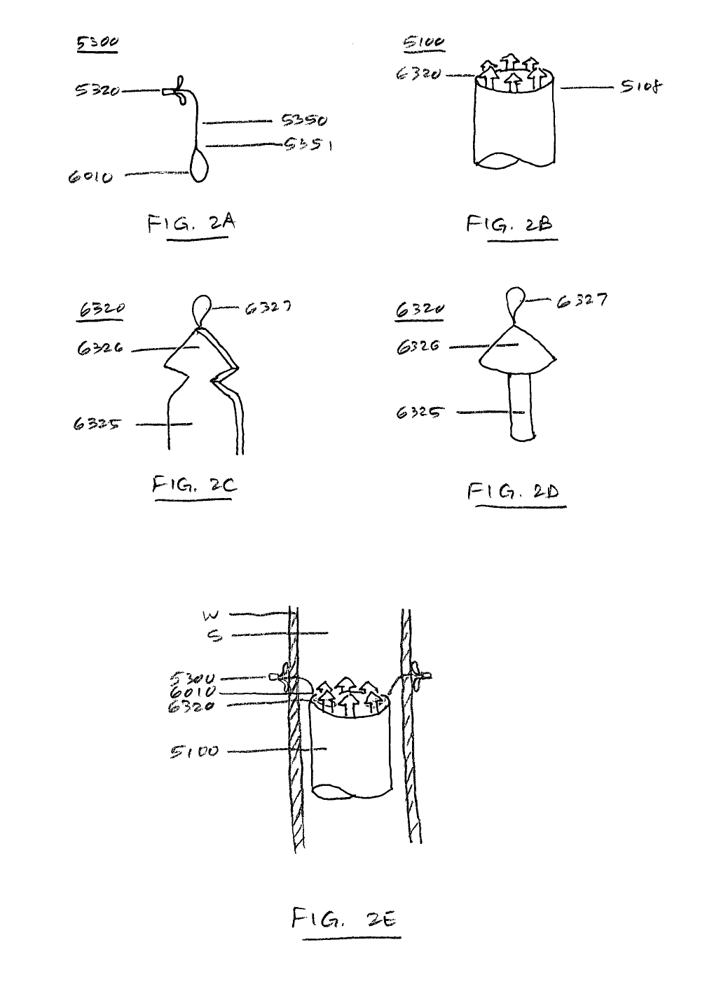

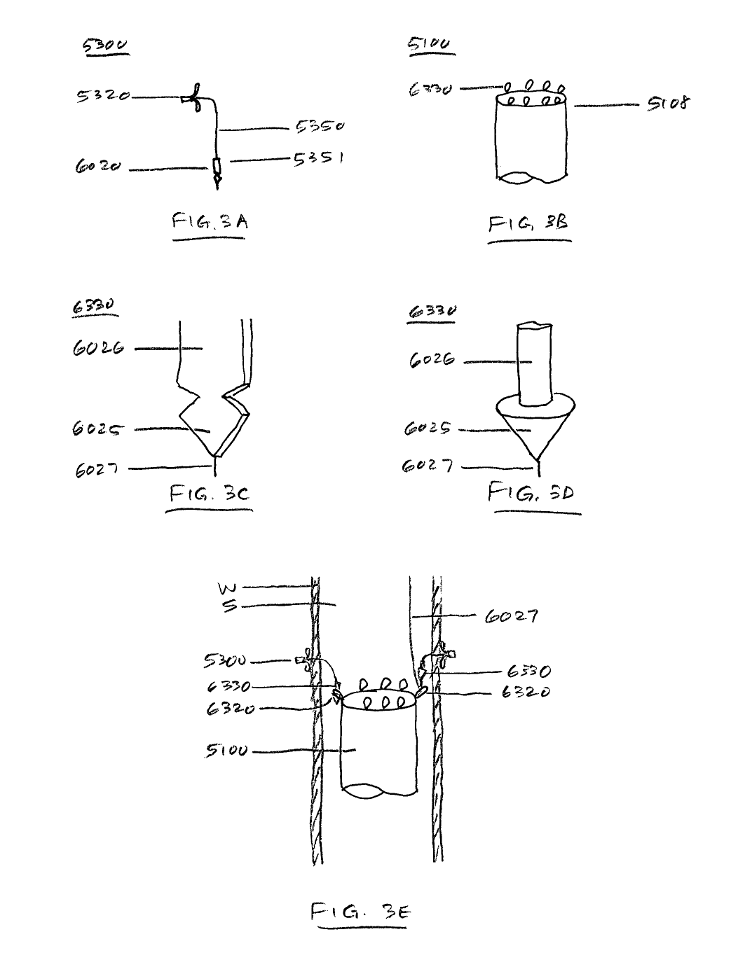

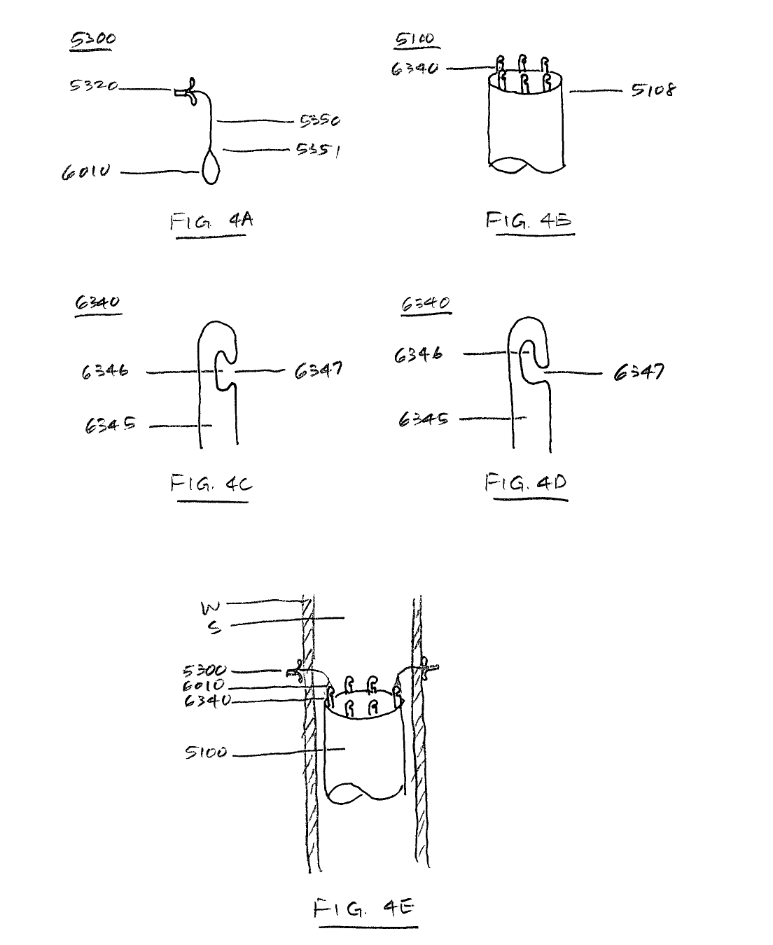

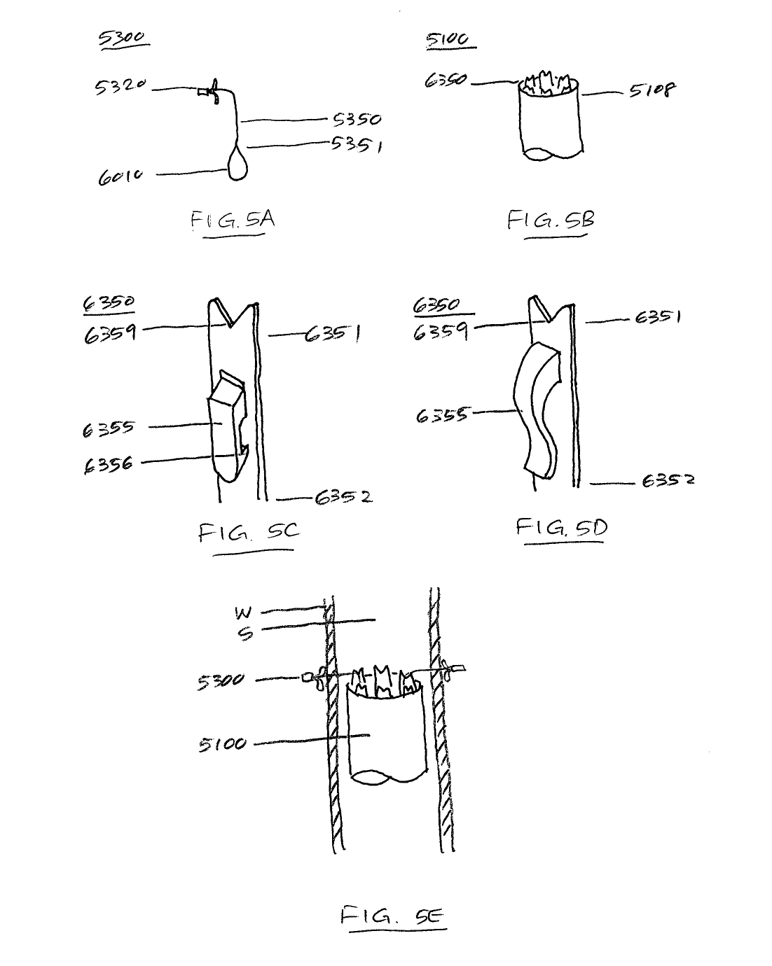

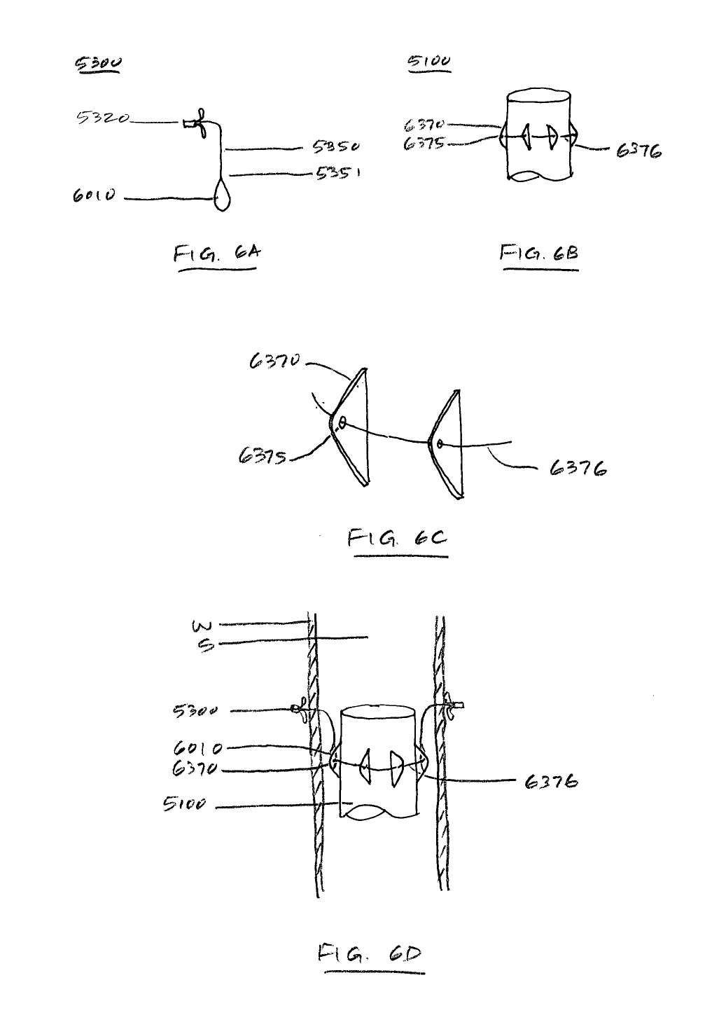

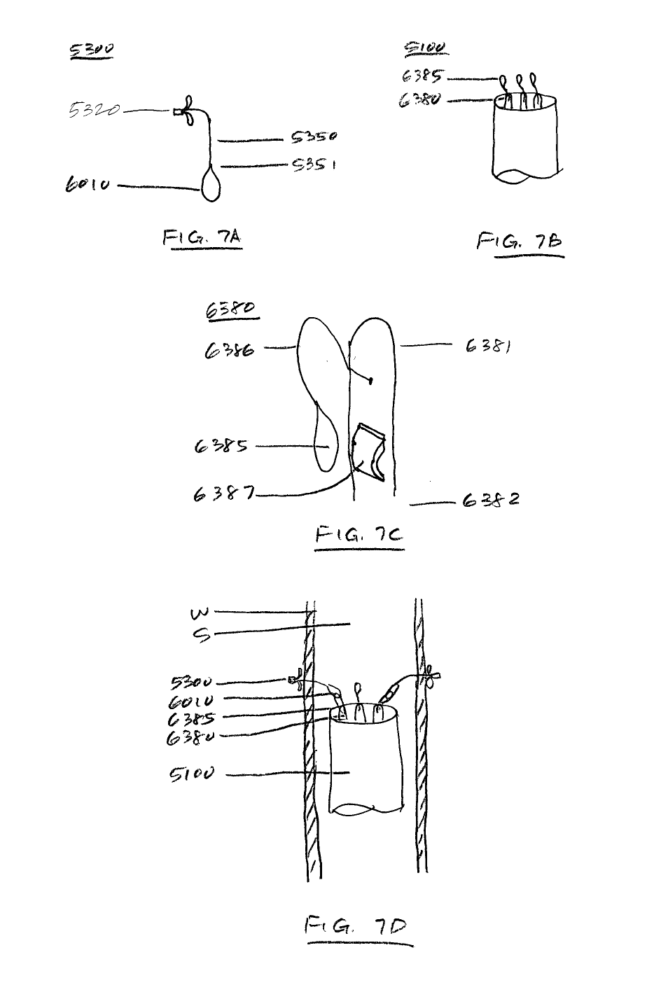

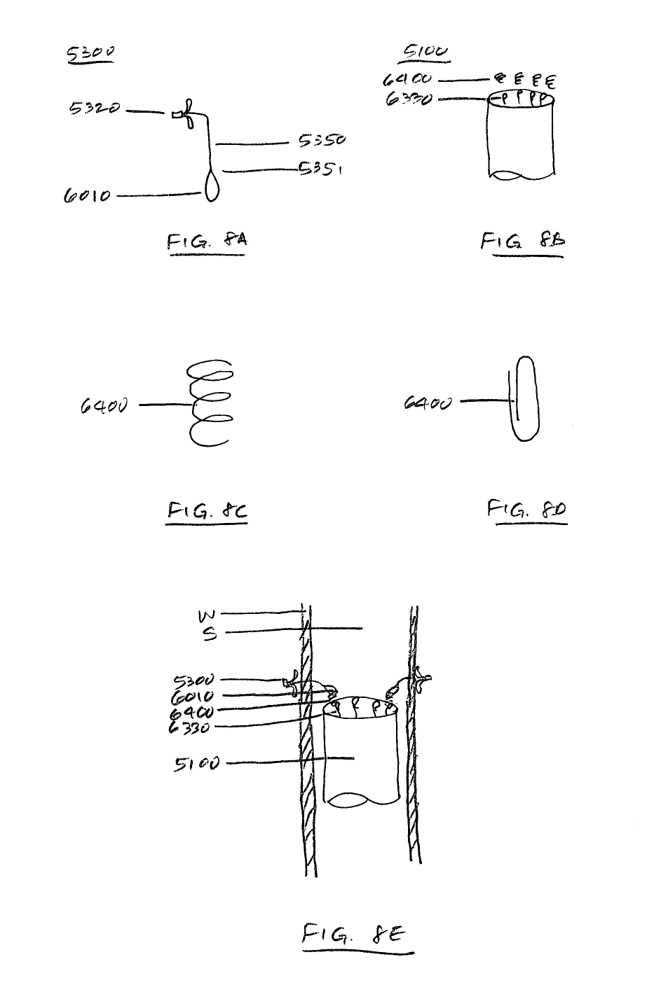

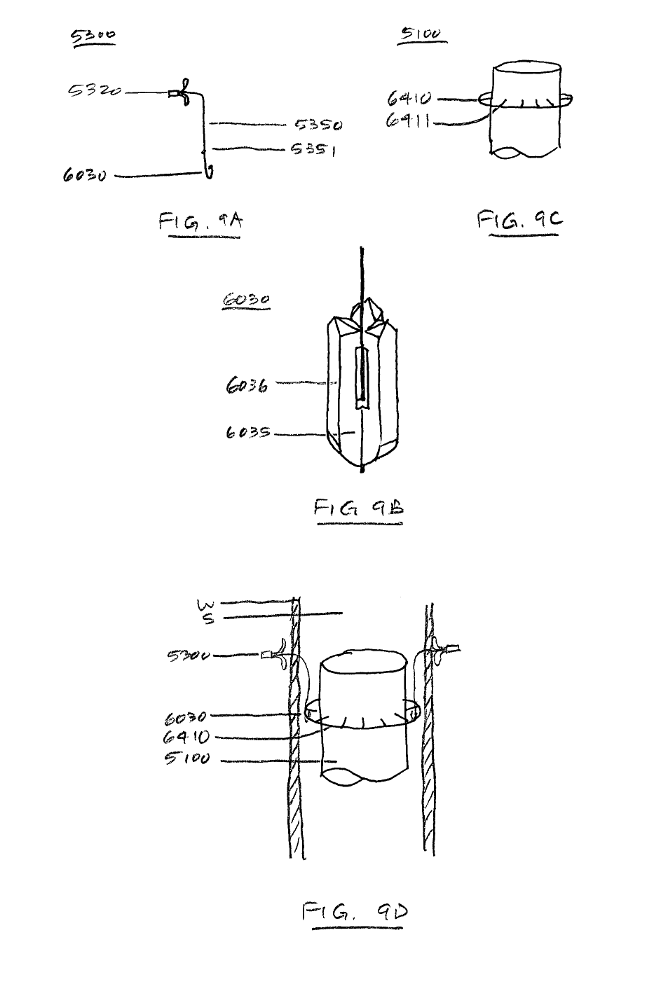

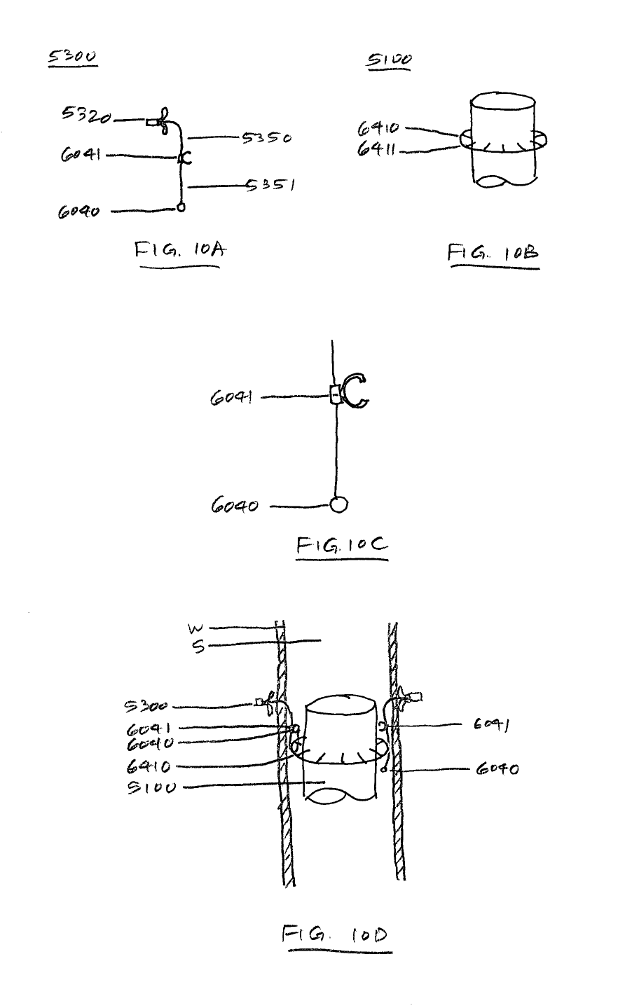

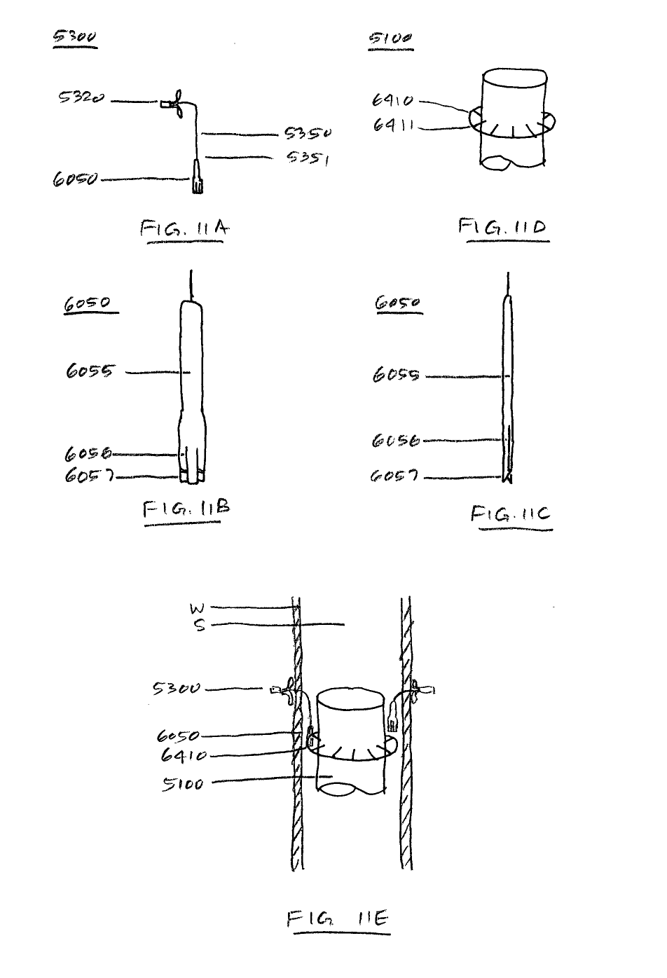

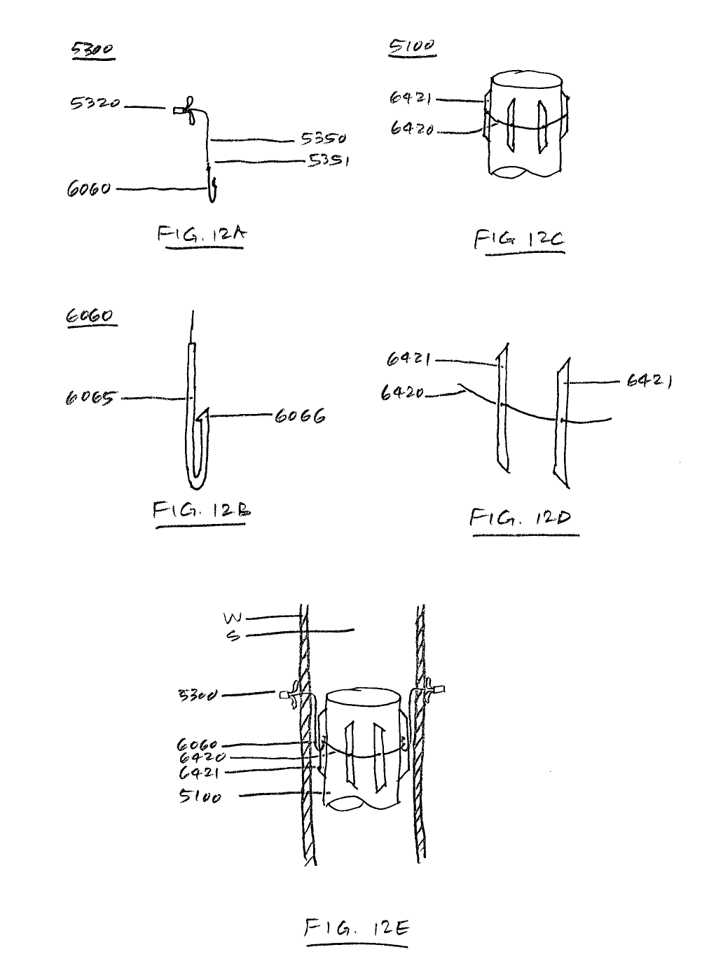

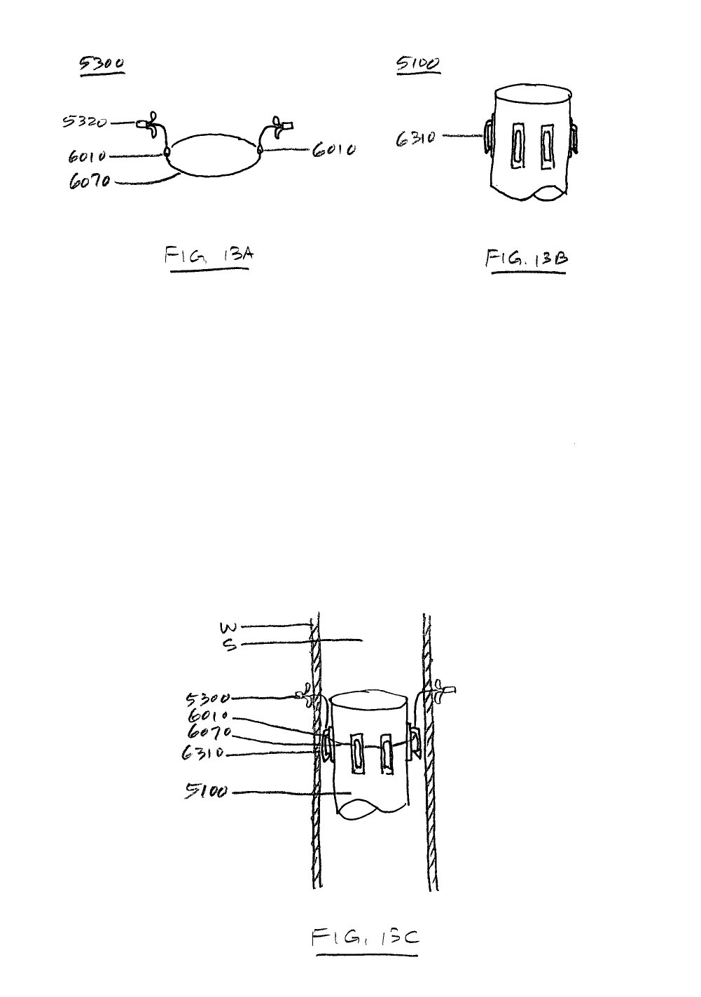

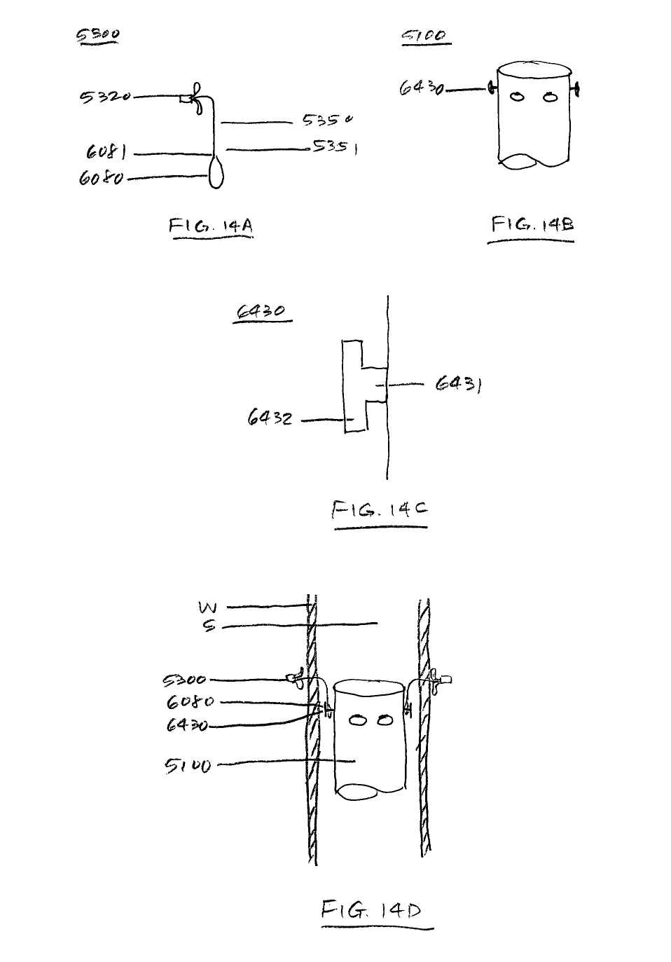

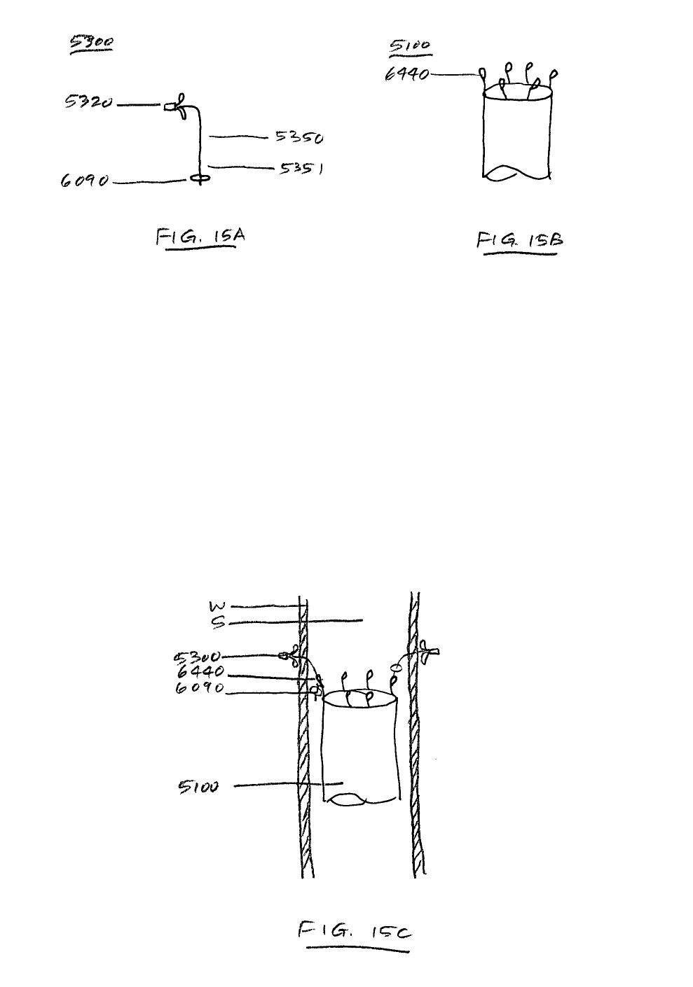

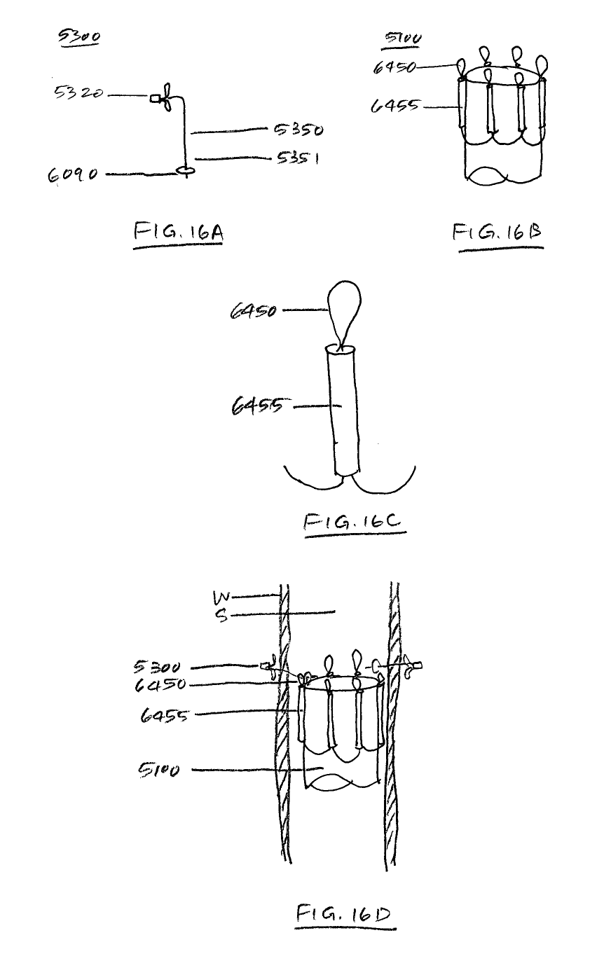

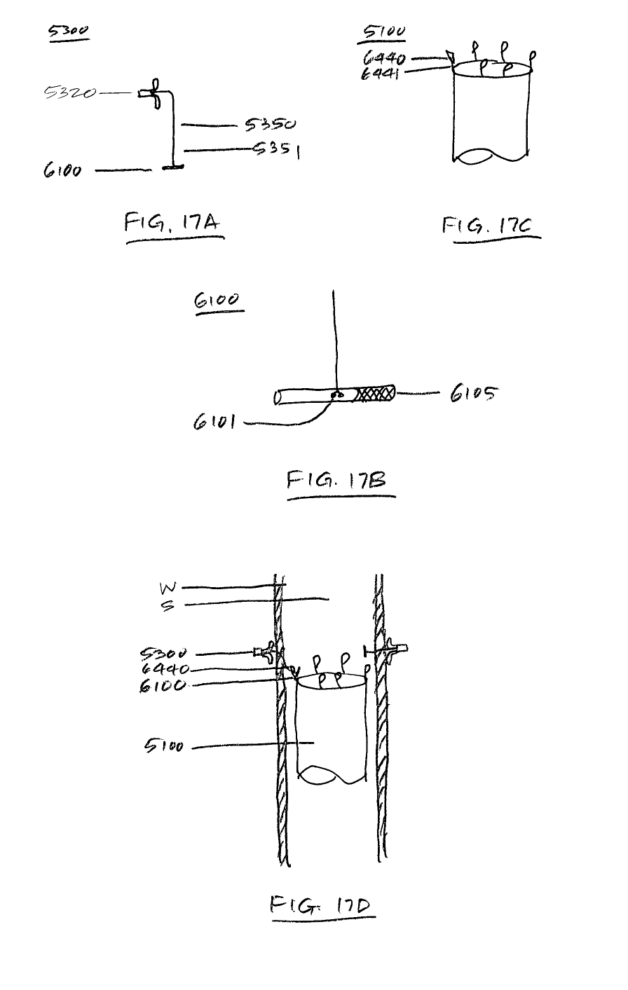

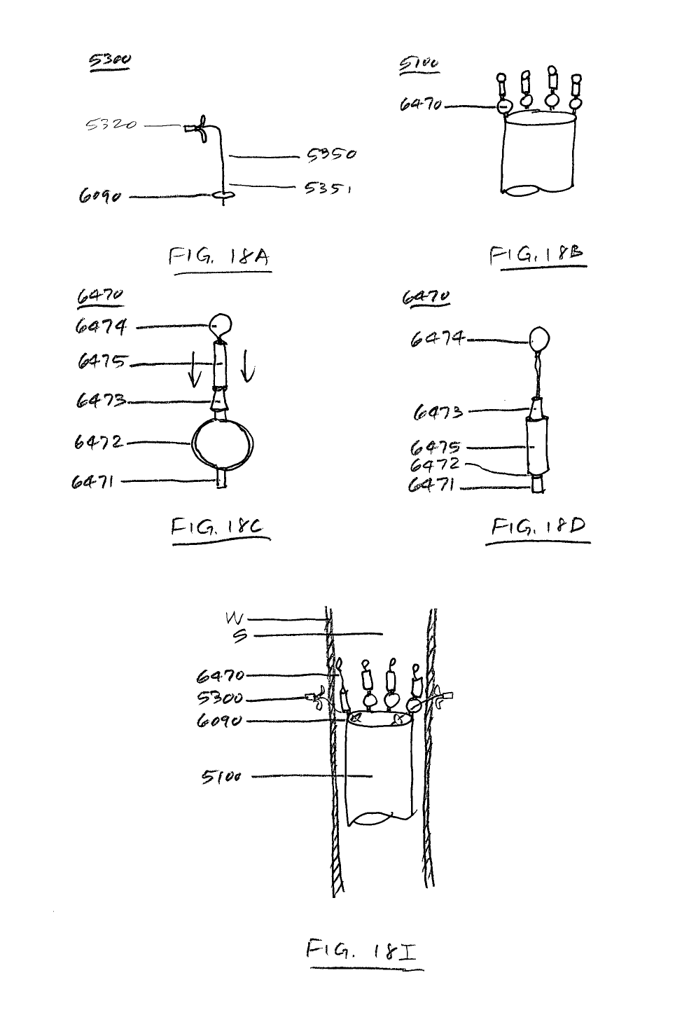

FIGS. 1A-47B show various embodiments of devices and methods for delivering an anchored device 5100 using one or more tissue anchors 5300.

Tissue anchor 5300 may include an anchor coupling 5310, a distal retention element 5320, and a tension element 5350.

Anchor coupling 5310 is configured to be positioned within a bodily space. Anchor coupling 5310 may include a loop 6010, barb 6020, hook 6030, ball and socket 6040, fork 6050, clip 6060, halo 6070, cinching loop 6080, button 6090, T-tag 6100, ball 6110, knob 6120, insert 6130, pop cap 6140, pincer 6150, expanding device 6160, magnet 6170, suture lock 6180, ball 6200, pin 6210, tube 6220, clip 6230, clip 6240, knob 6250, barb 6260, ball 6270, folding member 6280, and/or any other suitable coupling or attachment device.

Distal retention element 5320 may be configured to be deployed on a distal side of a tissue wall defining a bodily space. Distal retention element 5320 may include a retention element as described in U.S. Pat. No. 8,070,743, U.S. patent application Ser. Nos. 12/137,473, 13/485,887, and 13/896,838, each which is incorporated by reference in its entirety, or any other suitable retention element.

Tension element 5350 couples anchor coupling 5310 to distal retention element 5320. Proximal portion 5351 of tension element 5350 may be fixedly or adjustably coupled to anchor coupling 5310. Distal portion 5352 of tension element 5350 may be fixedly or adjustably coupled to distal retention element 5320. Tension element 5350 may be configured to pass through a tissue wall. Tension element 5350 may have a reduced width or thickness in order to decrease the size of the hole through a tissue wall, which may lower the likelihood of infection or other response. In one embodiment, tension element 5350 may have a diameter of approximately 0.2 to 0.5 mm. Tension element 5350 may be elastic or inelastic. Tension element 5350 may include a suture, wire, superelastic polymer, or other suitable material or device. Tension element 5350 may be coated or treated with an antibiotic agent. Alternatively, tension element 5350 may include an ultrathin coated stent that is stretchable and presents no interstitial spaces to surrounding tissue.

Tissue anchors 5300 may be delivered using the anchor delivery devices and methods described in U.S. patent application Ser. No. 13/485,887 and 13/896,838, each of which is incorporated by reference in its entirety, or any other suitable devices and/or methods. Tissue anchors 5300 may be delivered without first being coupled to anchored device 5100.

Anchored device 5100 may include a proximal portion 5101, a distal portion 5102, a lumen 5103, a longitudinal axis 5104, an interior surface 5106, an exterior surface 5107, a proximal edge 5108, and one or more device couplings 5130.

Anchored device 5100 may be any device configured to be attached at least partially within a bodily space. For example, anchored device 5100 may be a gastrointestinal cuff, gastrointestinal sleeve, gastrointestinal bypass device, GERD device, or other device configured to be attached at least partially within the esophagus, stomach, and/or intestine. Anchored device 5100 may be generally cylindrical, conical, or any other suitable configuration or shape.

Device coupling 5130 may be configured to be removably coupled to anchor coupling 5310 and/or tension element 5350. Alternatively, device coupling 5130 may be configured to be irremovably coupled to anchor coupling 5310 and/or tension element 5350. Device coupling 5130 may be coupled to anchored device 5100 as a separate element glued, stitched, welded, or otherwise attached to anchored device 5100. Alternatively, device coupling 5130 may be coupled to anchored device 5100 by being formed integrally with one or more elements of anchored device 5100. Device coupling 5130 may include a clip 6310, barb 6320, loop 6330, hitch 6340, strut 6350, fin 6370, strut 6380, loop coupling 6400, halo 6410, halo 6420, knob 6430, cinching loop 6440, cinching loop 6450, cinching loop 6470, strut 6500, strut 6520, strut 6540, strut 6560, ring 6580, strut 6590, block 6610, strut 6630, strut 6650, strut 6670, strut 6690, strut 6710, strut 6730, cleats 6750, strut 6760, hole 6780, magnet 6790, strut 6800, socket 6820, clip 6830, pin 6840, pin 6850, pin 6860, clip 6870, hole 6880, socket 6890, and/or any other suitable coupling or attachment device.

Anchored device 5100 may be delivered using endoscopic tools or any other suitable devices and/or methods. Anchored device 5100 may be delivered after tissue anchor 5300 is delivered. Alternatively, anchored device 5100 may be delivered before tissue anchor 5300, or coupled to tissue anchor 5300 and delivered at the same time as tissue anchor 5300.

Thus, in one embodiment, one or more tissue anchors 5300 may be delivered first, and then anchored device 5100 may be delivered second. Device coupling 5130 of anchored device 5100 may then be coupled to anchor coupling 5310 of tissue anchor 5300. This may ease delivery of tissue anchors 5300 and anchored device 5100 by dividing the procedure into two steps. Also, because anchored device 5100 does not encumber or otherwise interfere with tissue anchors 5300 during delivery of tissue anchors 5300, this may enhance placement accuracy of tissue anchors 5300. The use of device coupling 5130 and anchor coupling 5310 may allow anchored device 5100 to be removably coupled to tissue anchor 5300, which allows for anchored device 5100 to be replaced with a similar or different device. The option to place tissue anchors 5300 first may also allow at least a portion of any slack in tension element 5350 to be removed or taken up before anchored device 5100 is coupled to tissue anchors 5300.

FIG. 1A shows one embodiment of a tissue anchor 5300 including a loop 6010. FIG. 1B shows one embodiment of an anchored device 5100 including one or more clips 6310. FIGS. 1C-1D show enlarged views of two embodiments of clip 6310.

Loop 6010 may be formed from proximal portion 5351 of tension element 5350. Loop 6010 may be secured with one or more knots, adhesives, or other suitable ways.

Clip 6310 may include a finger 6311 with a barb 6316. Clip 6310 may also include a base 6317 configured to be coupled to anchored device 5100. Clip 6310 may be coupled to exterior surface 5107 of anchored device 5100. Alternatively, clip 6310 may be coupled to interior surface 5106 or proximal edge 5108 of anchored device 5100. Clip 6310 may be oriented substantially longitudinally, or in any orientation.

FIG. 1E shows one embodiment of a method for attaching anchored device 5100 within a bodily space S. One or more tissue anchors 5300 are delivered through the tissue wall W. Anchored device 5100 is delivered into the bodily space S. Loop 6010 is coupled to clip 6310.

Each loop 6010 may be coupled to one or more clips 6310. Each clip 6310 may be coupled to one or more loops 6010. To remove or exchange anchored device 5100, loops 6010 may be uncoupled from clips 6310.

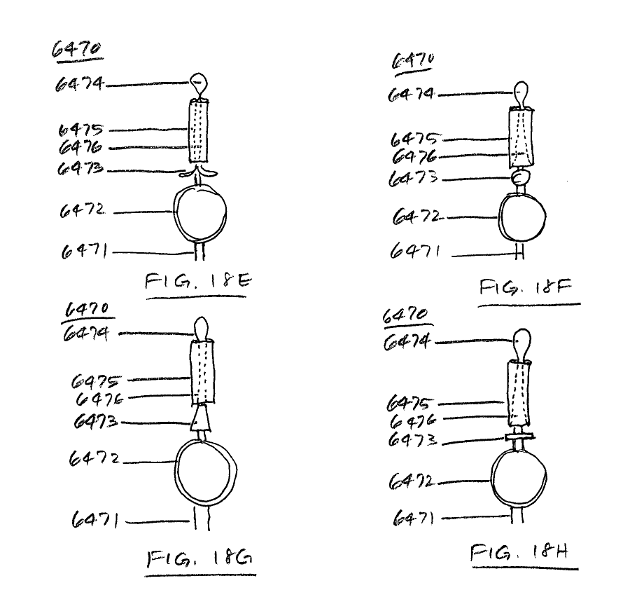

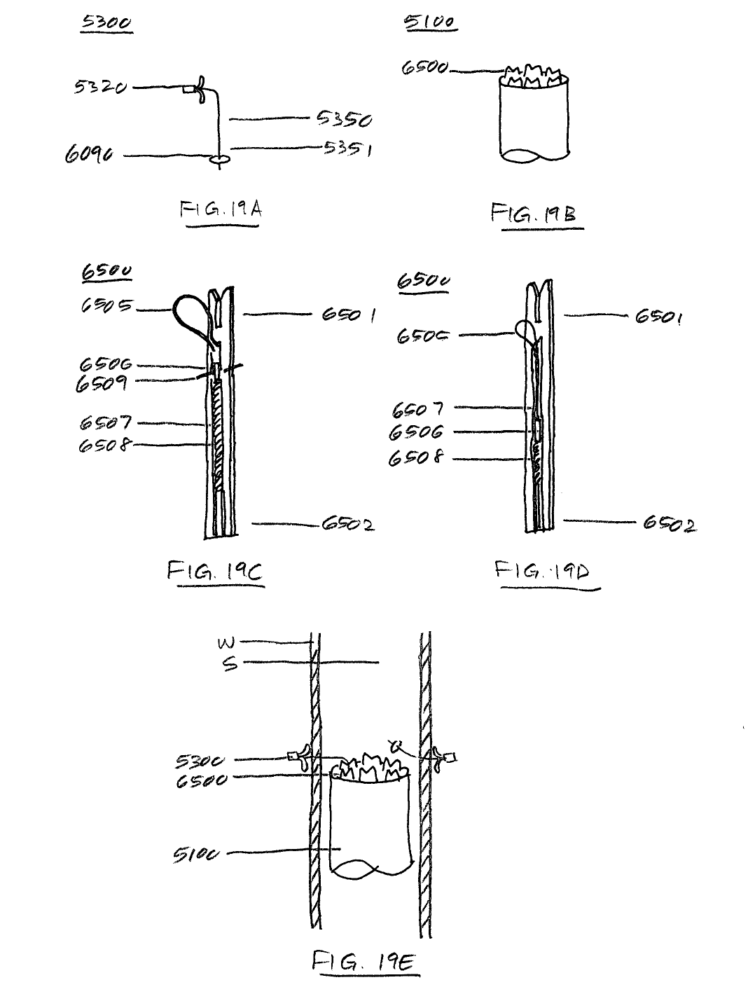

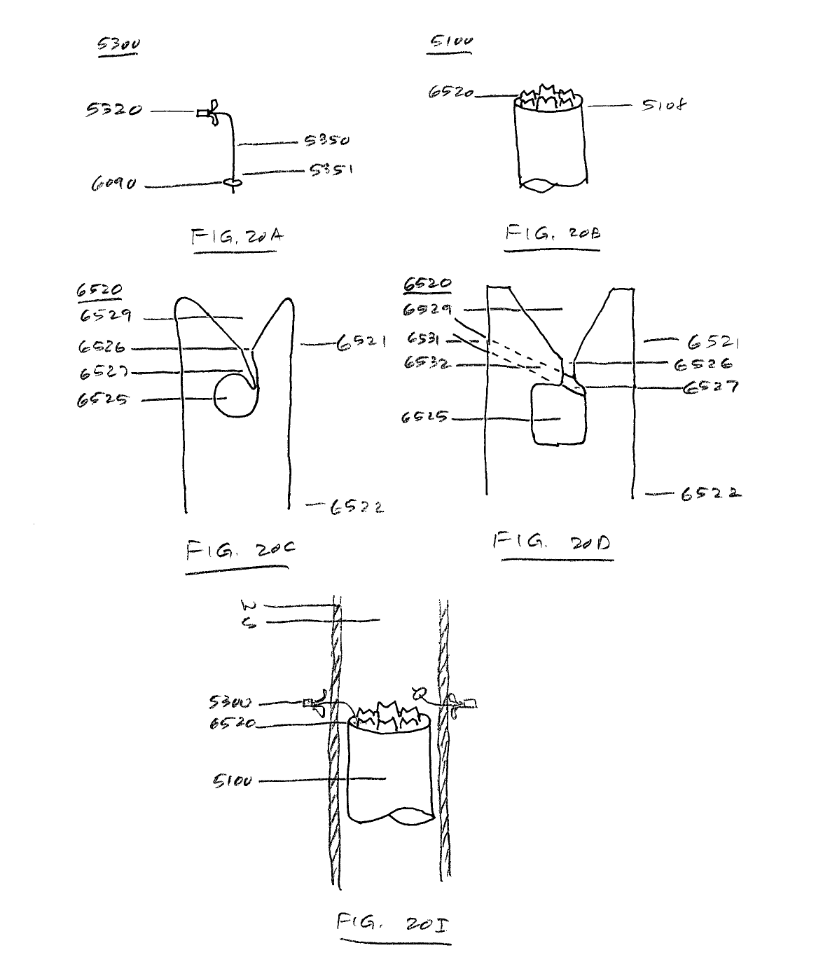

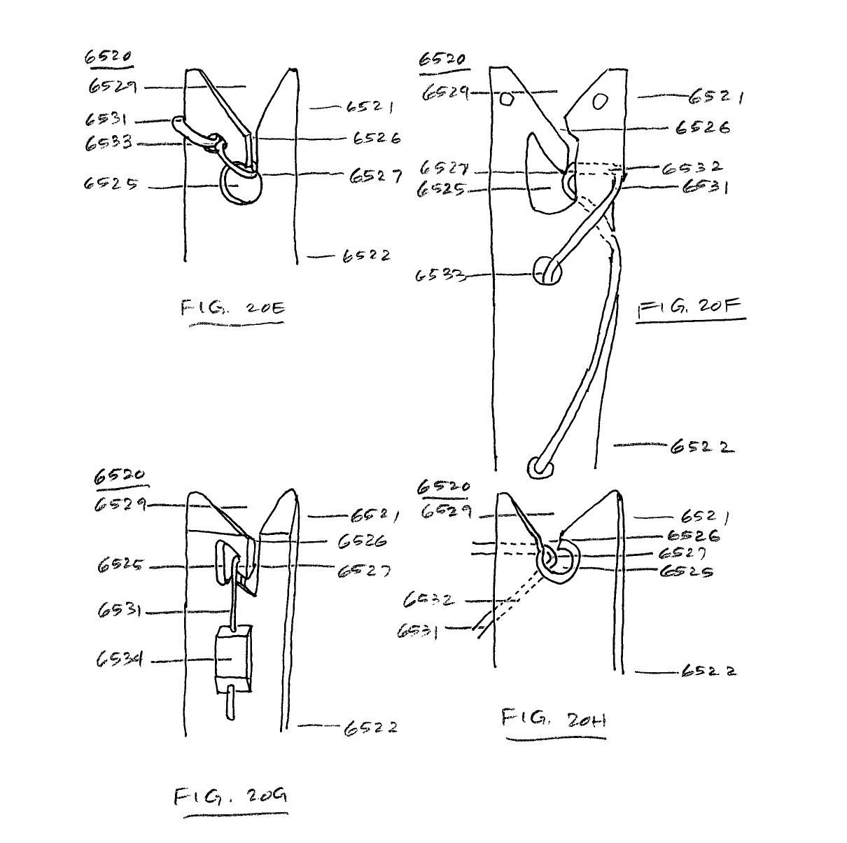

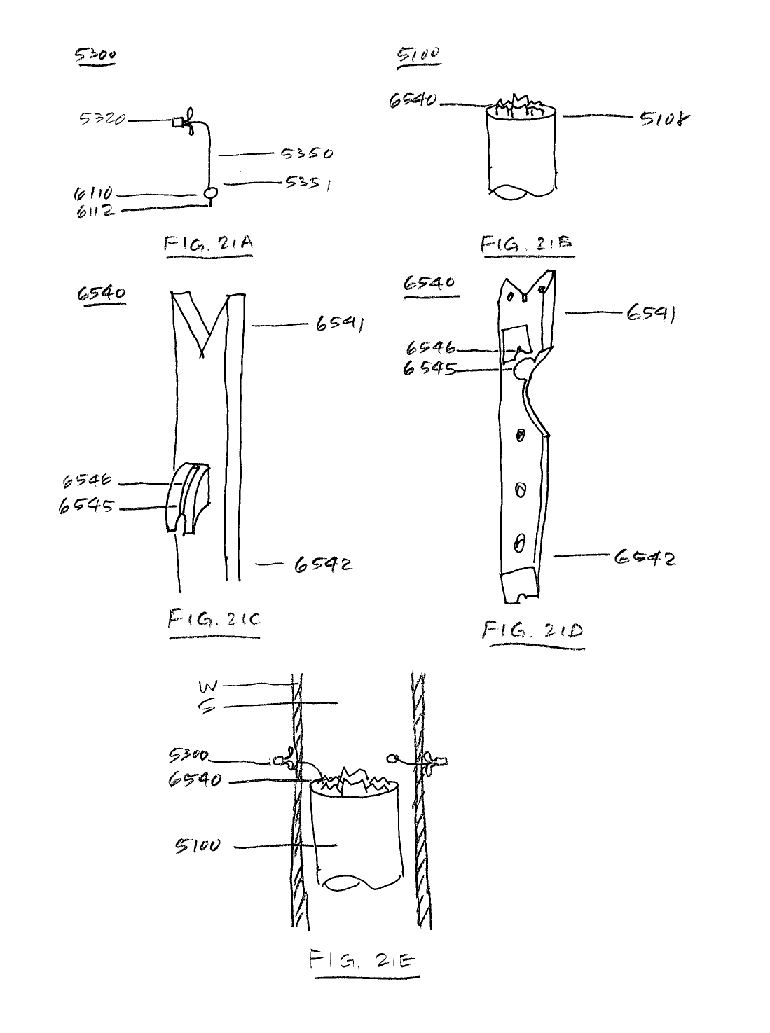

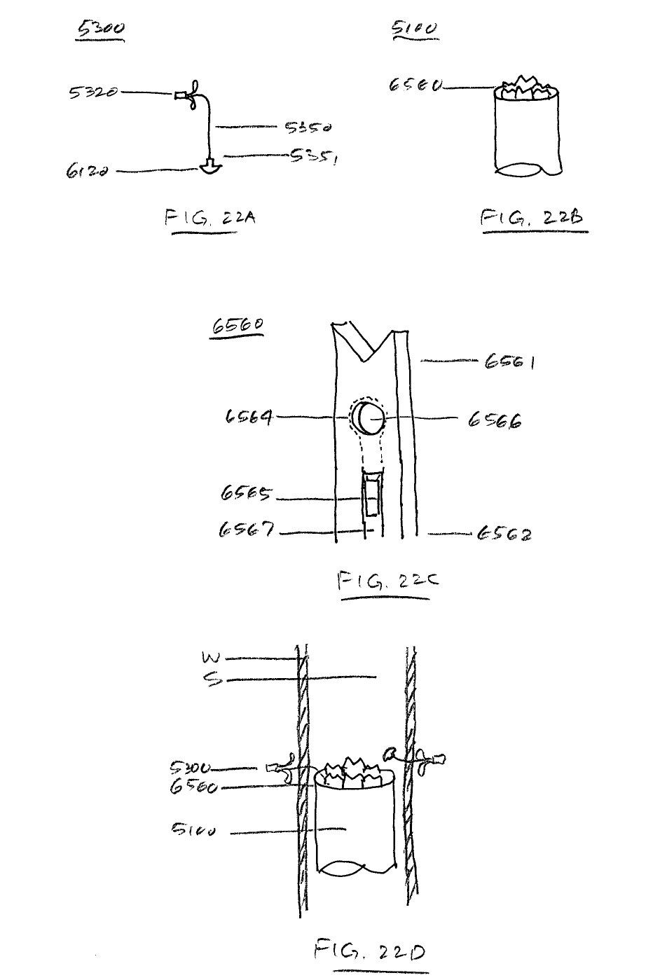

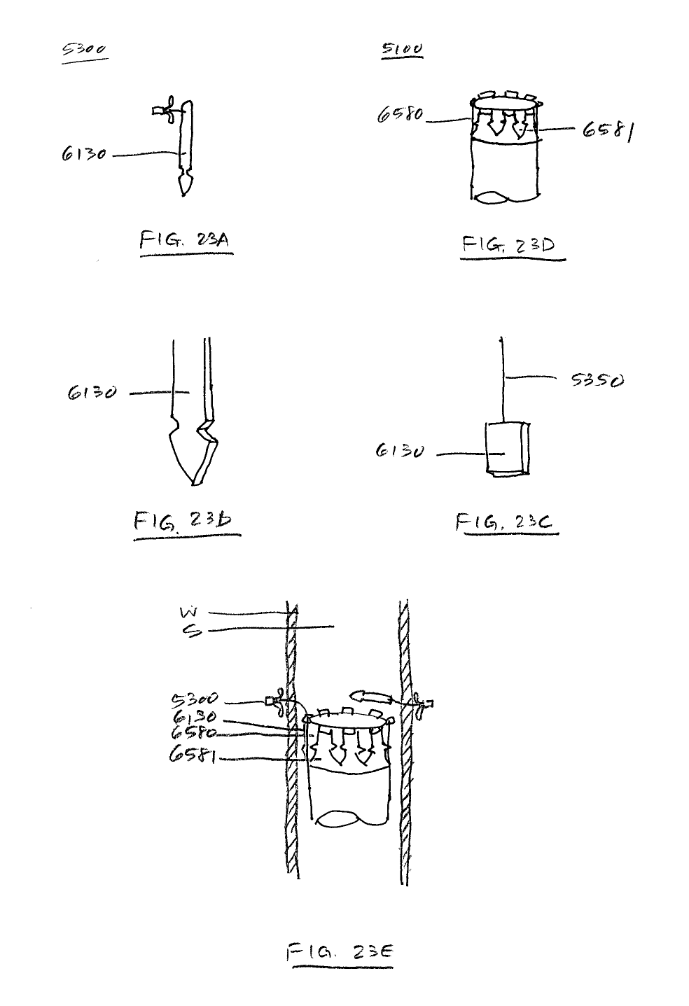

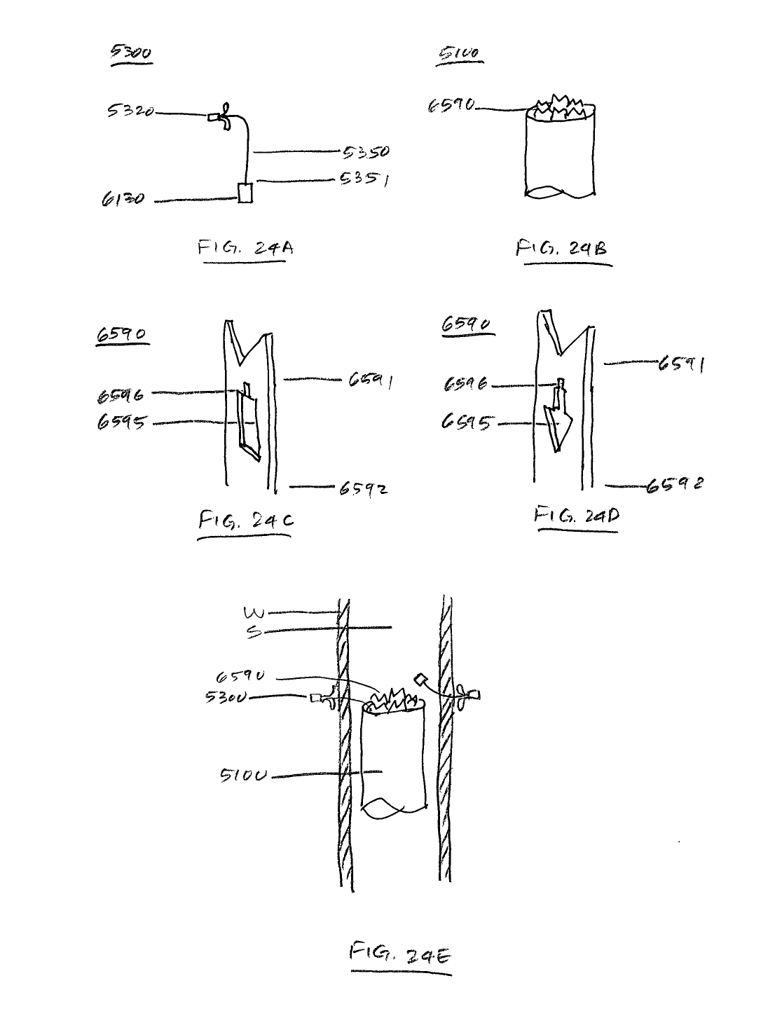

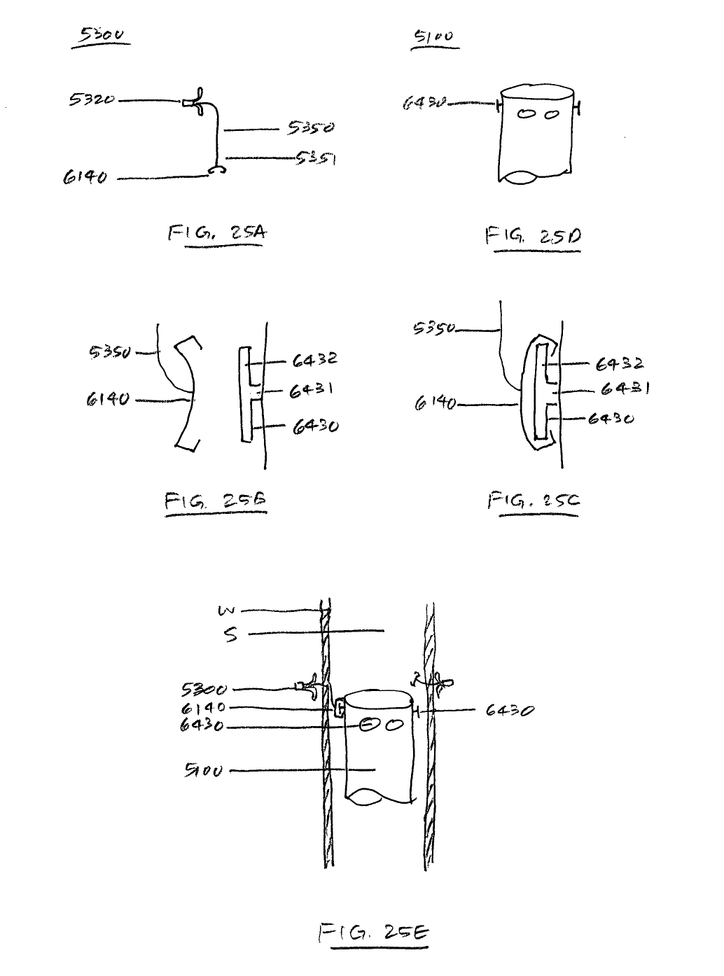

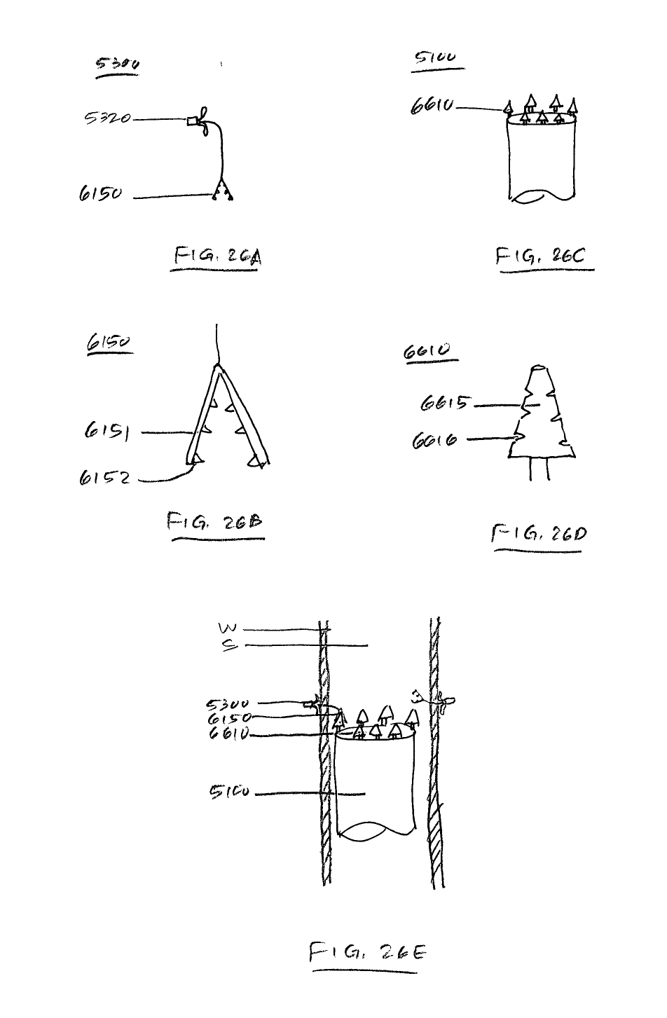

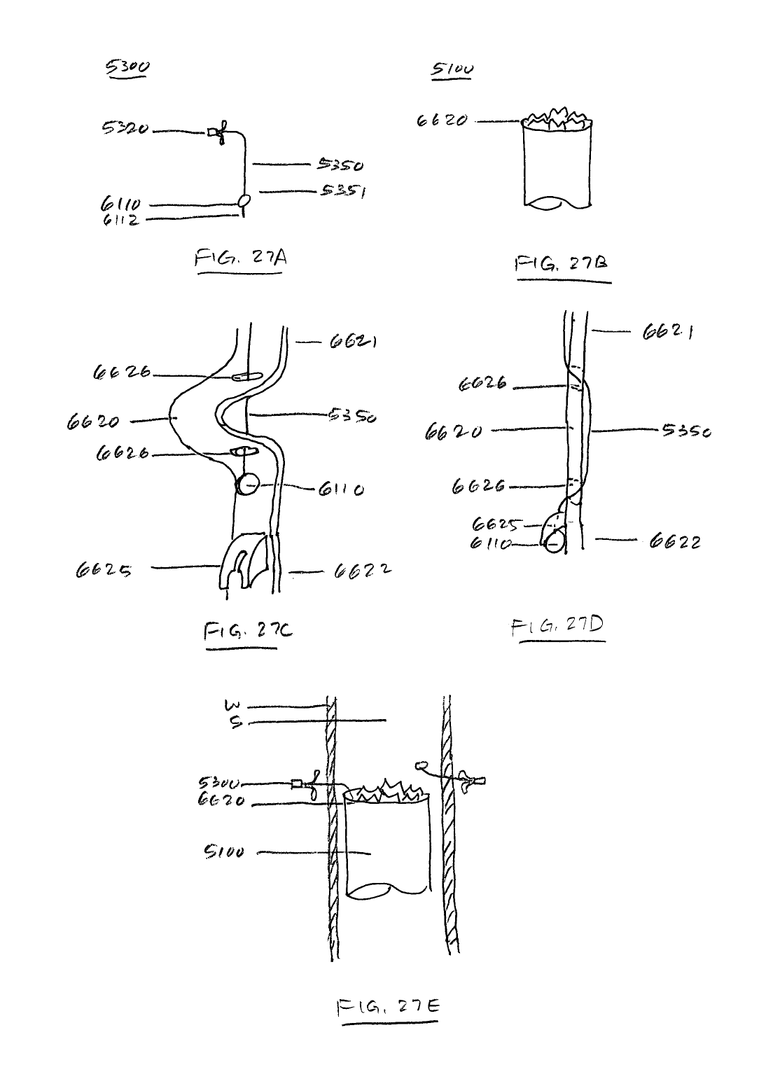

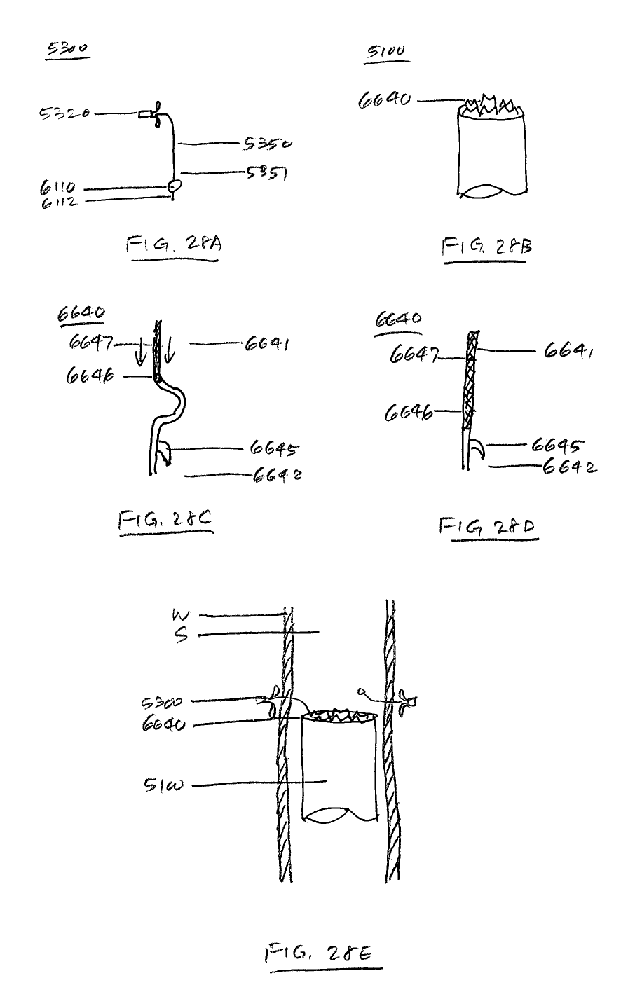

FIG. 2A shows one embodiment of a tissue anchor 5300 including a loop 6010. FIG. 2B shows one embodiment of an anchored device 5100 including one or more barbs 6320. FIGS. 2C-2D show enlarged views of two embodiments of barb 6320.

Loop 6010 may be formed from proximal portion 5351 of tension element 5350. Loop 6010 may be secured with one or more knots, adhesives, or other suitable ways.

Barb 6320 may include a stem 6325 and a cap 6326. Cap 6326 may be wider than stem 6325, and may have a generally conical or triangular shape. Barb 6320 may be made of a pliable yet semi-rigid material such as rubber, foam, or other suitable material. Barb 6320 may also include a pull tab 6327 coupled to cap 6326. Barb 6320 may be coupled to proximal edge 5108 of anchored device 5100. Alternatively, barb 6320 may be coupled to interior surface 5106 or exterior surface 5107 of anchored device 5100.

FIG. 2E shows one embodiment of a method for attaching anchored device 5100 within a bodily space S. One or more tissue anchors 5300 are delivered through the tissue wall W. Anchored device 5100 is delivered into the bodily space S. Cap 6326 is squeezed through loop 6010 to couple barb 6320 to loop 6010.

Each barb 6320 may be coupled to one or more loops 6010. To remove or exchange anchored device 5100, loops 6010 may be uncoupled from barbs 6320.

FIG. 3A shows one embodiment of a tissue anchor 5300 including a barb 6020. FIGS. 3B-3C show enlarged views of two embodiments of barb 6020. FIG. 3D shows one embodiment of an anchored device 5100 including one or more loops 6330.

Barb 6020 may include a stem 6025 and a cap 6026. Cap 6026 may be wider than stem 6025, and may have a generally conical or triangular shape. Barb 6020 may be made of a pliable yet semi-rigid material such as rubber, foam, or other suitable material. Barb 6020 may also include a guide line 6027 coupled to cap 6026. Guide line 6027 may be long enough to extend out of the bodily space.

Loop 6330 may be made of suture, wire, plastic, or other suitable material. Loop 6330 may be coupled to proximal edge 5108 of anchored device 5100. Alternatively, loop 6330 may be coupled to interior surface 5106 or exterior surface 5107 of anchored device 5100. Loop 6330 may be biased in toward or away from longitudinal axis 5104.

FIG. 3E shows one embodiment of a method for attaching anchored device 5100 within a bodily space S. One or more tissue anchors 5300 are delivered through the tissue wall W. Anchored device 5100 is delivered into the bodily space S. Loop 6330 may be “parachuted” down over guide line 6027 and over cap 6026. Cap 6026 is squeezed through loop 6330 to couple barb 6020 to loop 6330. Guide line 6027 may then be removed.

Each barb 6020 may be coupled to one or more loops 6330. To remove or exchange anchored device 5100, barbs 6020 may be uncoupled from loops 6330.

FIG. 4A shows one embodiment of a tissue anchor 5300 including a loop 6010. FIG. 4B shows one embodiment of an anchored device 5100 including one or more hitches 6340. FIGS. 4C-4D show enlarged views of two embodiments of hitch 6340.

Loop 6010 may be formed from proximal portion 5351 of tension element 5350. Loop 6010 may be secured with one or more knots, adhesives, or other suitable ways.

Hitch 6340 may include a body 6345 having a hole 6346 with an entry 6347. Entry 6347 may be narrow enough so that loop 6010 may be inserted through entry 6347, but is not easily removed from hole 6346. Hitch 6340 may be coupled to proximal edge 5108 of anchored device 5100. Alternatively, hitch 6340 may be coupled to interior surface 5106 or exterior surface 5107 of anchored device 5100.

FIG. 4E shows one embodiment of a method for attaching anchored device 5100 within a bodily space S. One or more tissue anchors 5300 are delivered through the tissue wall W. Anchored device 5100 is delivered into the bodily space S. Loop 6010 is coupled to hitch 6340. Loop 6010 may be wrapped one or more times around hitch 6340 through hole 6346.

Each loop 6010 may be coupled to one or more hitches 6340. Each hitch 6340 may be coupled to one or more loops 6010. To remove or exchange anchored device 5100, loops 6010 may be uncoupled from hitches 6340.

FIG. 5A shows one embodiment of a tissue anchor 5300 including a loop 6010. FIG. 5B shows one embodiment of an anchored device 5100 including one or more struts 6350. FIGS. 5C-5D show enlarged views of two embodiments of strut 6350.

Loop 6010 may be formed from proximal portion 5351 of tension element 5350. Loop 6010 may be secured with one or more knots, adhesives, or other suitable ways.

Strut 6350 includes a proximal portion 6351 and a distal portion 6352. Strut 6350 includes a clip 6355. Clip 6355 may include a barb 6356. Clip 6355 may be oriented substantially longitudinally, or in any orientation. Strut 6350 may include a notch 6359 at proximal portion 6351. Notch 6350 may be positioned at or near proximal edge 5108 of anchored device 5100. Strut 6350 may be coupled to interior surface 5106 of anchored device 5100.

FIG. 5E shows one embodiment of a method for attaching anchored device 5100 within a bodily space S. One or more tissue anchors 5300 are delivered through the tissue wall W. Anchored device 5100 is delivered into the bodily space S. Loop 6010 is coupled to clip 6355. Tension element 5350 may be placed in notch 6359.

Each loop 6010 may be coupled to one or more clips 6355. Each clip 6355 may be coupled to one or more loops 6010. To remove or exchange anchored device 5100, loops 6010 may be uncoupled from clips 6355.

FIG. 6A shows one embodiment of a tissue anchor 5300 including a loop 6010. FIG. 6B shows one embodiment of an anchored device 5100 including one or more fins 6370. FIG. 6C shows an enlarged view of one embodiment of fin 6370.

Loop 6010 may be formed from proximal portion 5351 of tension element 5350. Loop 6010 may be secured with one or more knots, adhesives, or other suitable ways.

Fin 6370 may include a hole 6375. A loop retention element 6376 may be threaded through holes 6375 of one or more fins 6370. Loop retention element 6376 may include a suture, wire, pins, or other suitable devices. Loop retention element 6376 may be separate or integral to fin 6370. Fin 6370 may be coupled to exterior surface 5107 of anchored device 5100. Alternatively, fin 6370 may be coupled to interior surface 5106 of anchored device 5100. Fin 6370 may be oriented substantially longitudinally, or in any orientation.

FIG. 6D shows one embodiment of a method for attaching anchored device 5100 within a bodily space S. One or more tissue anchors 5300 are delivered through the tissue wall W. Anchored device 5100 is delivered into the bodily space S. Loop 6010 is placed over fin 6370. Loop retention element 6376 is threaded through hole 6375 and secured.

Each loop 6010 may be coupled to one or more fins 6370. Each fin 6370 may be coupled to one or more loops 6010. To remove or exchange anchored device 5100, loop retention element 6376 may be cut or removed.

FIG. 7A shows one embodiment of a tissue anchor 5300 including a loop 6010. FIG. 7B shows one embodiment of an anchored device 5100 including one or more struts 6380. FIG. 7C shows an enlarged view of one embodiment of strut 6380.

Loop 6010 may be formed from proximal portion 5351 of tension element 5350. Loop 6010 may be secured with one or more knots, adhesives, or other suitable ways.

Strut 6380 includes a proximal portion 6381 and a distal portion 6382. Strut 6380 includes a loop 6385 with a tension element 6386. Strut 6380 may also include a loop retention element 6387. Loop retention element 6387 may include a hook, clip, or other suitable device. Strut 6380 may be coupled to exterior surface 5107 of anchored device 5100. Alternatively, strut 6380 may be coupled to interior surface 5106 of anchored device 5100.

FIG. 7D shows one embodiment of a method for attaching anchored device 5100 within a bodily space S. One or more tissue anchors 5300 are delivered through the tissue wall W. Anchored device 5100 is delivered into the bodily space S. Loop 6385 of strut 6380 is threaded through loop 6010 of tissue anchor 5300 and then coupled to loop retention element 6387.

Each loop 6010 may be coupled to one or more loops 6385. Each loop 6385 may be coupled to one or more loops 6010. To remove or exchange anchored device 5100, loops 6385 may be cut or uncoupled from loop retention elements 6387.

FIG. 8A shows one embodiment of a tissue anchor 5300 including a loop 6010. FIG. 8B shows one embodiment of an anchored device 5100 including a loop 6330 and a loop coupling 6400. FIGS. 8C-8D show enlarged views of two embodiments of loop coupling 6400.

Loop 6010 may be formed from proximal portion 5351 of tension element 5350. Loop 6010 may be secured with one or more knots, adhesives, or other suitable ways.

Loop 6330 of anchored device 5100 may be made of suture, wire, plastic, or other suitable material. Loop 6330 may be coupled to proximal edge 5108 of anchored device 5100. Alternatively, loop 6330 may be coupled to interior surface 5106 or exterior surface 5107 of anchored device 5100.

Loop coupling 6400 is configured to couple loop 6010 and loop 6330. Loop coupling 6400 may include a coil, clip, or other suitable device.

FIG. 8E shows one embodiment of a method for attaching anchored device 5100 within a bodily space S. One or more tissue anchors 5300 are delivered through the tissue wall W. Anchored device 5100 is delivered into the bodily space S. Loop 6010 is coupled to loop 6330 with a loop coupling 6400.

Each loop 6010 may be coupled to one or more loops 6330. Each loop 6330 may be coupled to one or more loops 6010. To remove or exchange anchored device 5100, loops 6330 may be cut or uncoupled from loop couplings 6400.

FIG. 9A shows one embodiment of a tissue anchor 5300 including a hook 6030. FIG. 9B shows an enlarged view of one embodiment of hook 6030. FIG. 9C shows one embodiment of an anchored device 5100 including a halo 6410.

Hook 6030 may include one or more prongs 6036 coupled to a body 6035. Prongs 6036 may be radially arranged, which may reduce the need to rotate or orient hook 6030 when coupling to halo 6410.

Halo 6410 may be coupled to anchored device 5100 by standoffs 6411. Halo 6410 may be made of suture, wire, plastic, or other suitable material. Halo 6410 may be coupled to exterior surface 5107 of anchored device 5100. Alternatively, halo 6410 may be coupled to interior surface 5106 or proximal edge 5108 of anchored device 5100.

FIG. 9D shows one embodiment of a method for attaching anchored device 5100 within a bodily space S. One or more tissue anchors 5300 are delivered through the tissue wall W. Anchored device 5100 is delivered into the bodily space S. Hook 6030 is coupled to halo 6410.

To remove or exchange anchored device 5100, halo 6410 may be cut or uncoupled from hooks 6030.

FIG. 10A shows one embodiment of a tissue anchor 5300 including a ball 6040 and a socket 6041. FIG. 10B shows an enlarged view of one embodiment of ball 6040 and socket 6141. FIG. 10C shows one embodiment of an anchored device 5100 including a halo 6410.

Ball 6040 is configured to be coupled to socket 6041. Ball 6040 may be coupled to socket 6041 with an interference fit or a snap fit. Socket 6041 may be coupled to tension element 5350 distally to ball 6040. Socket 6041 may be slidably coupled to tension element 5350 to allow adjustment.

Halo 6410 may be coupled to anchored device 5100 by standoffs 6411. Halo 6410 may be made of suture, wire, plastic, or other suitable material. Halo 6410 may be coupled to exterior surface 5107 of anchored device 5100. Alternatively, halo 6410 may be coupled to interior surface 5106 or proximal edge 5108 of anchored device 5100.

FIG. 10D shows one embodiment of a method for attaching anchored device 5100 within a bodily space S. One or more tissue anchors 5300 are delivered through the tissue wall W. Anchored device 5100 is delivered into the bodily space S. Ball 6040 is passed around halo 6410 and coupled to socket 6041.

To remove or exchange anchored device 5100, halo 6410 may be cut or balls 6040 uncoupled from sockets 6041.

FIG. 11A shows one embodiment of a tissue anchor 5300 including a fork 6050. FIGS. 11B-11C show enlarged views of one embodiment of fork 6050. FIG. 11D shows one embodiment of an anchored device 5100 including a halo 6410.

Fork 6050 may include a body 6055 and a plurality of tines 6056. Tines 6141 may include barbs 6057.

Halo 6410 may be coupled to anchored device 5100 by standoffs 6411. Halo 6410 may be made of suture, wire, plastic, or other suitable material. Halo 6410 may be coupled to exterior surface 5107 of anchored device 5100. Alternatively, halo 6410 may be coupled to interior surface 5106 or proximal edge 5108 of anchored device 5100.

FIG. 11E shows one embodiment of a method for attaching anchored device 5100 within a bodily space S. One or more tissue anchors 5300 are delivered through the tissue wall W. Anchored device 5100 is delivered into the bodily space S. Fork 6050 is coupled to halo 6410 by sliding tines 6141 onto halo 6410.

To remove or exchange anchored device 5100, halo 6410 may be cut or uncoupled from forks 6050.

FIG. 12A shows one embodiment of a tissue anchor 5300 including a clip 6060. FIG. 12B shows an enlarged view of one embodiment of clip 6060. FIG. 12C shows one embodiment of an anchored device 5100 including a halo 6420. FIG. 12D shows an enlarged view of halo 6420.

Clip 6060 may include a body 6065 and barb 6066. Clip 6060 may be configured to fit between guides 6421 of halo 6420.

Halo 6420 may be coupled to anchored device 5100 by guides 6421. Halo 6420 may be made of suture, wire, plastic, or other suitable material. Guides 6421 may be spaced to fit clips 6060. Halo 6420 may be coupled to exterior surface 5107 of anchored device 5100. Alternatively, halo 6420 may be coupled to interior surface 5106 of anchored device 5100.

FIG. 12E shows one embodiment of a method for attaching anchored device 5100 within a bodily space S. One or more tissue anchors 5300 are delivered through the tissue wall W. Anchored device 5100 is delivered into the bodily space S. Clip 6060 is passed between guides 6421 and coupled to halo 6420.

To remove or exchange anchored device 5100, halo 6420 may be cut or uncoupled from clips 6060.

FIG. 13A shows one embodiment of a tissue anchor 5300 including a loop 6010 and a halo 6070. FIG. 13B shows one embodiment of an anchored device 5100 including one or more clips 6310.

Loop 6010 may be formed from proximal portion 5351 of tension element 5350. Loop 6010 may be secured with one or more knots, adhesives, or other suitable ways. Halo 6070 may be threaded through loops 6010 of a plurality of tissue anchors 5300. Halo 6070 may be made of suture, wire, plastic, or other suitable material.

Clip 6310 may include a barb 6316. Clip 6310 may include a base 6317 configured to be coupled to anchored device 5100. Clip 6310 may be coupled to exterior surface 5107 of anchored device 5100. Alternatively, clip 6310 may be coupled to interior surface 5106 or proximal edge 5108 of anchored device 5100. Clip 6310 may be oriented substantially longitudinally, or in any orientation.

FIG. 13C shows one embodiment of a method for attaching anchored device 5100 within a bodily space S. One or more tissue anchors 5300 are delivered through the tissue wall W. Anchored device 5100 is delivered into the bodily space S. Halo 6070 is threaded through loops 6010 and the ends of halo 6070 secured to each other or to one or more of loops 6010. Halo 6070 is coupled to clip 6310.

To remove or exchange anchored device 5100, halo 6070 may be cut or uncoupled from clips 6310.

FIG. 14A shows one embodiment of a tissue anchor 5300 including a cinching loop 6080. FIG. 14B shows one embodiment of an anchored device 5100 including one or more knobs 6430. FIG. 14C shows an enlarged view of one embodiment of knob 6430.

Cinching loop 6080 may be formed from proximal portion 5351 of tension element 5350. Cinching loop 6080 may be cinched, and may include a slip knot 6081.

Knob 6430 may include a stem 6431 and a cap 6432. Cap 6432 may be wider than stem 6431, and may be cylindrical, hemispherical, rectangular, conical, or any other suitable shape. Knob 6430 may be coupled to exterior surface 5107 of anchored device 5100. Alternatively, knob 6430 may be coupled to interior surface 5106 or proximal edge 5108 of anchored device 5100.

FIG. 14D shows one embodiment of a method for attaching anchored device 5100 within a bodily space S. One or more tissue anchors 5300 are delivered through the tissue wall W. Anchored device 5100 is delivered into the bodily space S. Cinching loop 6080 is placed over cap 6432 and cinched around stem 6431.

Each cinching loop 6080 may be coupled to one or more knobs 6430. Each knob 6430 may be coupled to one or more cinching loops 6080. To remove or exchange anchored device 5100, cinching loop 6080 may be loosened from knob 6430.

FIG. 15A shows one embodiment of a tissue anchor 5300 including a button 6090. FIG. 15B shows one embodiment of an anchored device 5100 including one or more cinching loops 6440.

Button 6090 may be round, square, bar-shaped, or any other suitable shape. Button 6090 may include one or more holes 6091 through which proximal portion 5351 of tension element 5350 may be coupled. Button 6090 may allow tension element 5350 to be adjusted in length.

Cinching loop 6440 may be made of suture, wire, plastic, or other suitable material. Cinching loop 6440 may be coupled to proximal edge 5108 of anchored device 5100. Alternatively, cinching loop 6440 may be coupled to interior surface 5106 or exterior surface 5107 of anchored device. Cinching loop 6440 may be cinched, and may include a slip knot 6441.

FIG. 15C shows one embodiment of a method for attaching anchored device 5100 within a bodily space S. One or more tissue anchors 5300 are delivered through the tissue wall W. Anchored device 5100 is delivered into the bodily space S. Button 6090 is passed through cinching loop 6440, and then cinching loop 6440 is cinched.

Each button 6090 may be coupled to one or more cinching loops 6440. Each cinching loop 6440 may be coupled to one or more buttons 6090. To remove or exchange anchored device 5100, cinching loop 6440 may be cut or loosened.

FIG. 16A shows one embodiment of a tissue anchor 5300 including a button 6090. FIG. 16B shows one embodiment of an anchored device 5100 including one or more cinching loops 6450. FIG. 16C shows an enlarged view of one embodiment of cinching loop 6450.

Button 6090 may be round, square, bar-shaped, or any other suitable shape. Button 6090 may include one or more holes 6201 through which proximal portion 5351 of tension element 5350 may be coupled. Button 6090 may allow tension element 5350 to be adjusted in length.

Cinching loop 6450 may be made of suture, wire, plastic, or other suitable material. Cinching loop 6450 may be coupled to interior surface 5106 or exterior surface 5107 of anchored device. Cinching loop 6450 includes a cinching tube 6455, and may be cinched by pulling through cinching tube 6455.

FIG. 16D shows one embodiment of a method for attaching anchored device 5100 within a bodily space S. One or more tissue anchors 5300 are delivered through the tissue wall W. Anchored device 5100 is delivered into the bodily space S. Button 6090 is passed through cinching loop 6450, and then cinching loop 6220 is cinched by pulling through cinching tube 6455.

Each button 6090 may be coupled to one or more cinching loops 6450. Each cinching loop 6450 may be coupled to one or more buttons 6090. To remove or exchange anchored device 5100, cinching loop 6450 may be cut or loosened.

FIG. 17A shows one embodiment of a tissue anchor 5300 including a T-tag 6100. FIG. 17B shows an enlarged view of one embodiment of T-tag 6100. FIG. 17C shows one embodiment of an anchored device 5100 including one or more cinching loops 6440.

T-tag 6100 may be magnetized or include a magnet 6105 at one end. T-tag 6100 may include one or more holes 6101 through which proximal portion 5351 of tension element 5350 may be coupled.

Cinching loop 6440 may be made of suture, wire, plastic, or other suitable material. Cinching loop 6440 may be coupled to proximal edge 5108 of anchored device 5100. Alternatively, cinching loop 6440 may be coupled to interior surface 5106 or exterior surface 5107 of anchored device. Cinching loop 6440 may be cinched, and may include a slip knot 6441.

FIG. 17D shows one embodiment of a method for attaching anchored device 5100 within a bodily space S. One or more tissue anchors 5300 are delivered through the tissue wall W. Anchored device 5100 is delivered into the bodily space S. T-tag 6230 is passed through cinching loop 6440 with the assistance of a magnet. Cinching loop 6440 is then cinched.

Each T-tag 6100 may be coupled to one or more cinching loops 6440. Each cinching loop 6440 may be coupled to one or more T-tags 6100. To remove or exchange anchored device 5100, cinching loop 6440 may be cut or loosened.

FIG. 18A shows one embodiment of a tissue anchor 5300 including a button 6090. FIG. 18B shows one embodiment of an anchored device 5100 including one or more cinching loops 6470. FIGS. 18C-18D show enlarged views of one embodiment of cinching loop 6470 in an uncinched and cinched configurations. FIG. 18E-18H shows various embodiments of cinching loop 6470.

Button 6090 may be coupled to proximal portion 5351 of tension element 5350. Button 6090 may be round, square, bar-shaped, or any other suitable shape. Button 6090 may include one or more holes 6091 through which proximal portion 5351 of tension element 5350 may be coupled. Button 6090 may allow tension element 5350 to be adjusted in length.

Cinching loop 6470 may include a device attachment 6471, a loop 6472, a retainer 6473, a pull tab 6474, and a cinching element 6475. Device attachment 6471 may be configured to couple a distal portion of loop 6472 to anchored device 5100. Loop 6472 may be made of suture, wire, plastic, or other suitable material. Retainer 6473 may be coupled to a proximal portion of loop 6472. Retainer 6473 may be spherical, conical, bar-shaped, or any other suitable configuration. Pull tab 6474 may be coupled to retainer 6473. Cinching element 6475 may be slidably coupled to pull tab 6474. Cinching element 6475 may include a tube having a lumen 6476 that is uniform or tapered. Cinching loop 6470 may be coupled to proximal edge 5108 of anchored device 5100. Alternatively, cinching loop 6470 may be coupled to interior surface 5106 or exterior surface 5107 of anchored device 5100.

FIG. 18I shows one embodiment of a method for attaching anchored device 5100 within a bodily space S. One or more tissue anchors 5300 are delivered through the tissue wall W. Anchored device 5100 is delivered into the bodily space S. Button 6090 is passed through loop 6472, and pull tab 6474 is held while cinching element 6475 is pulled over retainer 6473 to cinch loop 6472. Retainer 6473 keeps cinching element 6475 in place and prevents loop 6472 from loosening.

Each button 6090 may be coupled to one or more cinching loops 6470. Each cinching loop 6470 may be coupled to one or more buttons 6090. To remove or exchange anchored device 5100, cinching element 6475 may be pulled back over retainer 6473 to loosen loop 6242. Alternatively, loop 6472 may be cut.

FIG. 19A shows one embodiment of a tissue anchor 5300 including a button 6090. FIG. 19B shows one embodiment of an anchored device 5100 including one or more struts 6500. FIGS. 19C-19D show enlarged views of one embodiment of strut 6500 in an uncinched and cinched configurations.

Button 6090 may be coupled to proximal portion 5351 of tension element 5350. Button 6090 may be round, square, bar-shaped, or any other suitable shape. Button 6090 may include one or more holes 6091 through which proximal portion 5351 of tension element 5350 may be coupled. Button 6090 may allow tension element 5350 to be adjusted in length.

Strut 6500 includes a proximal portion 6501 and a distal portion 6502. Strut 6500 may include a loop 6505, a slider 6506, a channel 6507, and a spring 6508. Loop 6505 may be coupled to slider 6506. Loop 6505 may extend out of a proximal opening of channel 6507. Slider 6506 is slidably coupled to channel 6507. Spring 6508 is coupled to slider 6506, and biases slider 6506 distally to cinch loop 6505. A locking element 6509 such as a pin or a suture may be used to retain slider 6506 in a uncinched position. Strut 6500 may be coupled to interior surface 5106 or exterior surface 5107 of anchored device 5100.

FIG. 19E shows one embodiment of a method for attaching anchored device 5100 within a bodily space S. One or more tissue anchors 5300 are delivered through the tissue wall W. Anchored device 5100 is delivered into the bodily space S. Button 6090 is passed through loop 6505, and locking element 6509 is unlocked to release slider 6506 and cinch loop 6505.

Each loop 6505 may be coupled to one or more buttons 6090. To remove or exchange anchored device 5100, slider 6506 may be manipulated to loosen loop 6505. Alternatively, loop 6505 may be cut.

FIG. 20A shows one embodiment of a tissue anchor 5300 including a button 6090. FIG. 20B shows one embodiment of an anchored device 5100 including one or more struts 6520. FIGS. 20C-20H show enlarged views of various embodiments of strut 6520.

Button 6090 may be round, square, bar-shaped, or any other suitable shape. Button 6090 may include one or more holes 6091 through which proximal portion 5351 of tension element 5350 may be coupled. Button 6090 may allow tension element 5350 to be adjusted in length.

Strut 6520 includes a proximal portion 6521 and a distal portion 6522. Strut 6520 includes a hole 6525 with an entry 6526. Hole 6525 is smaller than button 6090. Hole 6525 may be shaped to help retain tension element 5350. Entry 6526 may be narrow enough so that tension element 5350 may be passed through entry 6526, but is not easily removed. Strut 6520 may include a retainer 6527 configured to allow tension element 5350 to pass through entry 6526 and into hole 6525, and to help retain tension element 5350 in hole 6525. Retainer 6527 may be flexible and formed integrally with strut 6520, as shown in FIG. 20C. Retainer 6527 may include a suture 6531 passed through one or more channels 6532 formed in strut 6520, as shown in FIGS. 20D, 20F, and 20H. Retainer 6527 may include a suture 6531 tied through one or more holes 6533 formed in strut 6520, as shown in FIGS. 20E-20F. Retainer 6527 may include a suture 6528 coupled to a block 6534 coupled to strut 6520, as shown in FIG. 20H. Alternatively, retainer 6527 may include a wire, clip, or other suitable device. Strut 6520 may include a notch 6529 at proximal portion 6510. Notch 6529 may be configured to guide tension element 5350 toward entry 6526. Notch 6529 may be positioned at or near proximal edge 5108 of anchored device 5100. Strut 6520 may be coupled to interior surface 5106 or exterior surface 5107 of anchored device 5100.

FIG. 20I shows one embodiment of a method for attaching anchored device 5100 within a bodily space S. One or more tissue anchors 5300 are delivered through the tissue wall W. Anchored device 5100 is delivered into the bodily space S. Tension element 5350 is guided by notch 6529 through entry 6526 and into hole 6525. Retainer 6527 flexes and allows tension element 5350 to pass through entry 6526 and into hole 6525.

Each strut 6520 may be coupled to one or more buttons 6090. To remove or exchange anchored device 5100, retainer 6527 may be held open, pulled out, cut, or broken to release button 6090.

FIG. 21A shows one embodiment of a tissue anchor 5300 including one or more balls 6110. FIG. 21B shows one embodiment of an anchored device 5100 including one or more struts 6540. FIGS. 21C-21D shows enlarged views of two embodiments of strut 6540.

Balls 6110 may include balls or knots along a length of tension element 5350. Balls 6110 may include a pull tab 6112 such as a loop or length of suture.

Strut 6540 includes a proximal portion 6541 and a distal portion 6542. Strut 6540 includes a socket 6545 configured to be coupled to ball 6110. Socket 6545 may be coupled to ball 6110 with an interference fit or snap fit. Socket 6545 may be at least partially formed by removing a portion of strut 6540. Strut 6540 may include a slot 6546 for tension element 5350. Strut 6540 may be coupled to interior surface 5106 or exterior surface 5107 of anchored device 5100.

FIG. 21E shows one embodiment of a method for attaching anchored device 5100 within a bodily space S. One or more tissue anchors 5300 are delivered through the tissue wall W. Anchored device 5100 is delivered into the bodily space S. Ball 6110 is coupled to socket 6545.

To remove or exchange anchored device 5100, ball 6110 may be uncoupled from socket 6545.

FIG. 22A shows one embodiment of a tissue anchor 5300 including a knob 6120. FIG. 22B shows one embodiment of an anchored device 5100 including one or more struts 6560. FIG. 22C shows an enlarged view of one embodiment of strut 6560.

Knob 6120 may include a stem 6121 and a cap 6122. Cap 6122 may be wider than stem 6121, and may be cylindrical, hemispherical, rectangular, conical, or any other suitable shape.

Strut 6560 includes a proximal portion 6561 and a distal portion 6562. Strut 6560 may include a loop 6564, a slider 6565, a hole 6566, and a channel 6567. Hole 6566 and channel 6567 are in communication. Loop 6564 is positioned within channel 6567 and at least partially encircles hole 6566. Loop 6564 is coupled to slider 6565. Slider 6565 is slidably coupled within channel 6567. Sliding slider 6565 within channel 6567 cinches and uncinches loop 6564 within hole 6566. Strut 6560 may be coupled to interior surface 5106 or exterior surface 5107 of anchored device 5100.

FIG. 22D shows one embodiment of a method for attaching anchored device 5100 within a bodily space S. One or more tissue anchors 5300 are delivered through the tissue wall W. Anchored device 5100 is delivered into the bodily space S. Knob 6120 is inserted through hole 6566, and then slider 6565 is slid within channel 6567 to cinch loop 6564.

To remove or exchange anchored device 5100, slider 6565 may be slid within channel 6567 to uncinch loop 6564. Knob 6120 may then be removed from hole 6566.

FIG. 23A shows one embodiment of a tissue anchor 5300 including an insert 6130. FIGS. 23B-23C show enlarged views of various embodiments of insert 6130. FIG. 23D shows one embodiment of an anchored device 5100 including an attachment ring 6580.

Insert 6130 may be barb-shaped, square, round, or any other suitable shape. Insert 6130 may be made of a substantially rigid material. Alternatively, insert 6130 may be made of a pliable yet semi-rigid material such as rubber, foam, or other suitable material.

Attachment ring 6580 may include one or more recesses 6581 configured to be coupled to insert 6130. Recess 6581 may be coupled to insert 6130 with an interference fit or snap fit. Attachment ring 6580 may be coupled to proximal edge 5108 of anchored device 5100. Alternatively, attachment ring 6580 may be coupled to interior surface 5106 or exterior surface 5107 of anchored device 5100.

FIG. 23E shows one embodiment of a method for attaching anchored device 5100 within a bodily space S. One or more tissue anchors 5300 are delivered through the tissue wall W. Anchored device 5100 is delivered into the bodily space S. Insert 6130 is coupled to recess 6581.

To remove or exchange anchored device 5100, inserts 6130 may be uncoupled from recess 6581.

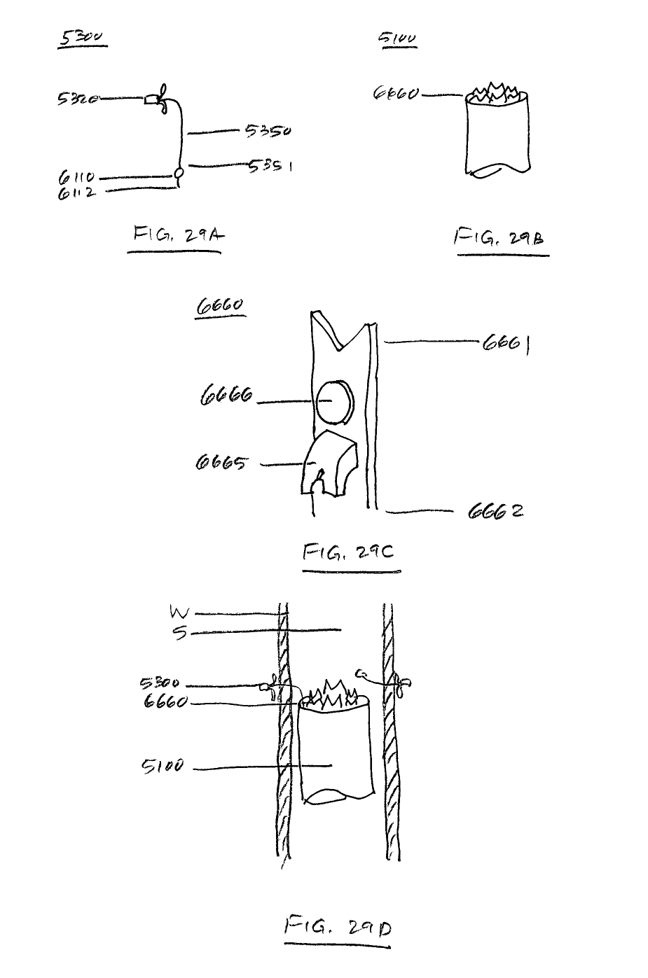

FIG. 24A shows one embodiment of a tissue anchor 5300 including an insert 6130. FIG. 24B shows one embodiment of an anchored device 5100 including one or more struts 6590. FIGS. 24C-24D show enlarged views of two embodiments of strut 6590.

Insert 6130 may be square, barb-shaped, round, or any other suitable shape. Insert 6130 may be made of a substantially rigid material. Alternatively, insert 6130 may be made of a pliable yet semi-rigid material such as rubber, foam, or other suitable material.

Strut 6590 includes a proximal portion 6591 and a distal portion 6592. Strut 6590 includes a recess 6595 configured to be coupled to insert 6130. Recess 6595 may be coupled to insert 6130 with an interference fit or snap fit. Strut 6590 may also include a slot 6596 for tension element 5350. Strut 6590 may be coupled to interior surface 5106 or exterior surface 5107 of anchored device 5100.

FIG. 24E shows one embodiment of a method for attaching anchored device 5100 within a bodily space S. One or more tissue anchors 5300 are delivered through the tissue wall W. Anchored device 5100 is delivered into the bodily space S. Insert 6130 is coupled to recess 6595.

To remove or exchange anchored device 5100, inserts 6130 may be uncoupled from recesses 6595.

FIG. 25A shows one embodiment of a tissue anchor 5300 including a pop cap 6140. FIGS. 25B-25C show enlarged views of one embodiment of pop cap 6140. FIG. 25D shows one embodiment of an anchored device 5100 including one or more knobs 6430.

Pop cap 6140 includes an open configuration as shown in FIG. 25B, and a closed configuration as shown in FIG. 25C.

Knob 6430 may include a stem 6431 and a cap 6432. Cap 6432 may be wider than stem 6431, and may be cylindrical, hemispherical, rectangular, conical, or any other suitable shape. Knob 6430 may be coupled to exterior surface 5107 of anchored device 5100. Alternatively, knob 6430 may be coupled to interior surface 5106 or proximal edge 5108 of anchored device 5100.

FIG. 25E shows one embodiment of a method for attaching anchored device 5100 within a bodily space S. One or more tissue anchors 5300 are delivered through the tissue wall W. Anchored device 5100 is delivered into the bodily space S. Edges of pop cap 6140 are pushed down over knob 6430 to couple pop cap 6140 to knob 6430.

To remove or exchange anchored device 5100, a center of pop cap 6140 may be pushed down, and/or edges of pop cap 6140 may be lifted up, to uncouple pop cap 6140 from knob 6430.

FIG. 26A shows one embodiment of a tissue anchor 5300 including a pincer 6150. FIG. 26B shows an enlarged view of one embodiment of pincer 6150. FIG. 26C shows one embodiment of an anchored device 5100 including one or more blocks 6610. FIG. 26D shows an enlarged view of one embodiment of block 6610.

Pincer 6150 may include jaws 6151 having one or more teeth 6152.

Block 6610 may include a body 6615 having one or more recesses 6616 configured to receive teeth 6152. Alternatively, block 6610 may be made of a soft material which teeth 6152 can penetrate. Block 6610 may be conical, cylindrical, spherical, or any other suitable shape. Block 6610 may be coupled to proximal edge 5108 of anchored device 5100. Alternatively, block 6610 may be coupled to interior surface 5106 or exterior surface 5107 of anchored device 5100.

FIG. 26E shows one embodiment of a method for attaching anchored device 5100 within a bodily space S. One or more tissue anchors 5300 are delivered through the tissue wall W. Anchored device 5100 is delivered into the bodily space S. Pincer 6150 is pushed down over block 6610 to couple pincer 6150 to block 6610.

To remove or exchange anchored device 5100, pincer 6150 may be pulled off of block 6610.

FIG. 27A shows one embodiment of a tissue anchor 5300 including one or more balls 6110. FIG. 27B shows one embodiment of an anchored device 5100 including one or more struts 6620. FIGS. 27C-27D show enlarged views of one embodiment of strut 6620.

Balls 6110 may include balls or knots along a length of tension element 5350. Balls 6110 may include a pull tab 6112 such as a loop or length of suture.

Strut 6620 includes a proximal portion 6621 and a distal portion 6622. Strut 6620 includes a socket 6625 configured to be coupled to ball 6110. Socket 6625 may be coupled to ball 6110 with an interference fit or snap fit. Strut 6620 includes two or more holes 6626 positioned at varying distances proximal to socket 6625. Holes 6626 are large enough for ball 6110 to fit through. Holes 6626 may be spaced to take up at least a portion of any slack in tension element 5350. Strut 6620 may have a flexed configuration, as shown in FIG. 27C, and an unflexed configuration, as shown in FIG. 27D. Strut 6620 may be coupled to interior surface 5106 of anchored device 5100.

FIG. 27E shows one embodiment of a method for attaching anchored device 5100 within a bodily space S. One or more tissue anchors 5300 are delivered through the tissue wall W. Anchored device 5100 is delivered into the bodily space S. Strut 6620 may be shortened by flexing strut 6620 in the vicinity of holes 6626. Ball 6110 is then passed through holes 6626 and coupled to socket 6625. Strut 6620 is then unflexed.

To remove or exchange anchored device 5100, ball 6110 may be uncoupled from socket 6625 and removed through holes 6626.

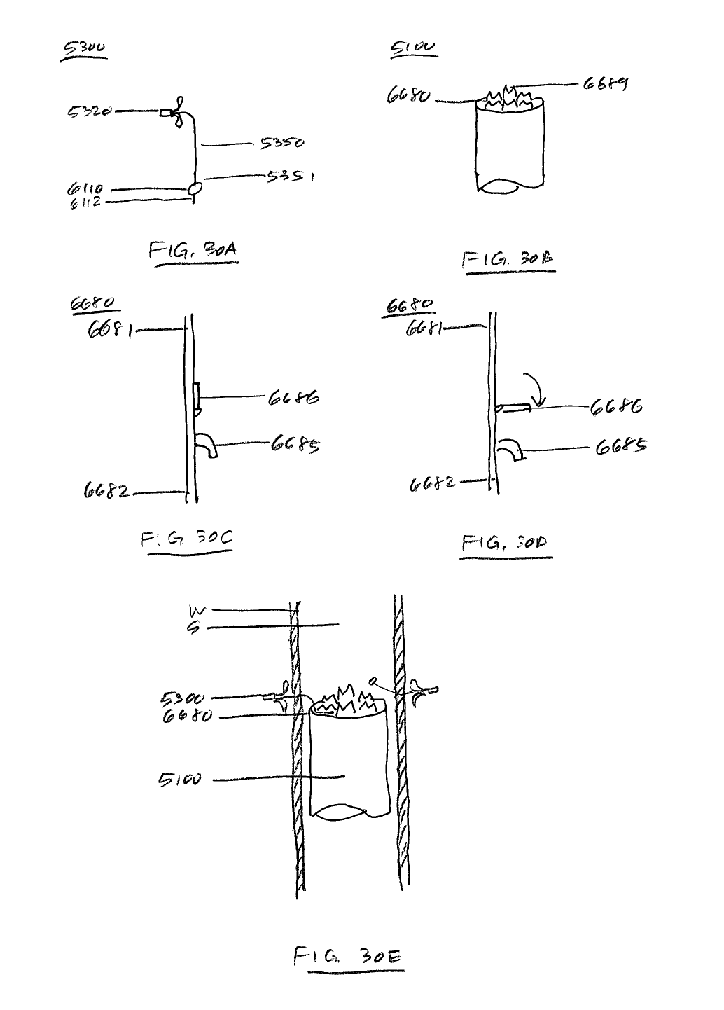

FIG. 28A shows one embodiment of a tissue anchor 5300 including one or more balls 6110. FIG. 28B shows one embodiment of an anchored device 5100 including one or more struts 6640. FIGS. 28C-28D show enlarged views of one embodiment of strut 6640.

Balls 6110 may include balls or knots along a length of tension element 5350. Balls 6110 may include a pull tab 6112 such as a loop or length of suture.

Strut 6640 includes a proximal portion 6641 and a distal portion 6642. Strut 6640 includes a socket 6645 configured to be coupled to ball 6110. Socket 6645 may be coupled to ball 6110 with an interference fit or snap fit. Strut 6640 is biased in a shortened or compressed configuration with one or more bends proximal to socket 6645, as shown in FIG. 28C. Strut 6640 includes a channel 6646 formed substantially longitudinally, and a rod 6647 slidably coupled to channel 6646. Rod 6647 is configured to slide within channel 6646 and lengthen or straighten strut 6640, as shown in FIG. 28D, which may take up at least a portion of any slack in tension element 5350. Strut 6640 may be coupled to interior surface 5106 of anchored device 5100.

FIG. 28E shows one embodiment of a method for attaching anchored device 5100 within a bodily space S. One or more tissue anchors 5300 are delivered through the tissue wall W. Anchored device 5100 is delivered into the bodily space S. Strut 6640 is in a shortened or compressed configuration. Ball 6110 is then coupled to socket 6645. Rod 6647 is slid within channel 6646 to lengthen or straighten strut 6640.

To remove or exchange anchored device 5100, ball 6110 may be uncoupled from socket 6645.

FIG. 29A shows one embodiment of a tissue anchor 5300 including one or more balls 6110. FIG. 29B shows one embodiment of an anchored device 5100 including one or more struts 6660. FIG. 29C shows an enlarged view of one embodiment of strut 6660.

Balls 6110 may include balls or knots along a length of tension element 5350. Balls 6110 may include a pull tab 6112 such as a loop or length of suture.

Strut 6660 includes a proximal portion 6661 and a distal portion 6662. Strut 6660 includes a socket 6665 configured to be coupled to ball 6110. Socket 6665 may be coupled to ball 6110 with an interference fit or snap fit. Strut 6660 includes a spool 6666 proximal to socket 6665. Spool 6666 may be fixed or rotating. Spool 6666 is configured to take up at least portion of any slack in tension element 5350. Strut 6660 may be coupled to interior surface 5106 of anchored device 5100.

FIG. 29D shows one embodiment of a method for attaching anchored device 5100 within a bodily space S. One or more tissue anchors 5300 are delivered through the tissue wall W. Anchored device 5100 is delivered into the bodily space S. Tension element 5350 may be wound one or more times around spool 6666. Ball 6110 is then coupled to socket 6665.

To remove or exchange anchored device 5100, ball 6110 may be uncoupled from socket 6665 and tension element 5350 unwound from spool 6666.

FIG. 30A shows one embodiment of a tissue anchor 5300 including one or more balls 6110. FIG. 30B shows one embodiment of an anchored device 5100 including one or more struts 6680. FIGS. 30C-30D show enlarged views of one embodiment of strut 6680.

Balls 6110 may include balls or knots along a length of tension element 5350. Balls 6110 may include a pull tab 6112 such as a loop or length of suture.

Strut 6680 includes a proximal portion 6681 and a distal portion 6682. Strut 6680 includes a socket 6685 configured to be coupled to ball 6110. Socket 6685 may be coupled to ball 6110 with an interference fit or snap fit. Strut 6680 includes a shelf 6686 proximal to socket 6685. Shelf 6686 folds down to a position substantially perpendicular to strut 6680, as shown in FIGS. 30C-30D. Shelf 6686 is configured to “tent” tension element 5350 and take up at least portion of any slack in tension element 5350. Strut 6680 may include a notch 6689 at proximal portion 6681. Notch 6689 may be positioned at or near proximal edge 5108 of anchored device 5100. Strut 6680 may be coupled to interior surface 5106 of anchored device 5100.

FIG. 30E shows one embodiment of a method for attaching anchored device 5100 within a bodily space S. One or more tissue anchors 5300 are delivered through the tissue wall W. Anchored device 5100 is delivered into the bodily space S. Ball 6110 is coupled to socket 6685. Tension element 5350 is passed over shelf 6686 and placed in notch 6689. Shelf 6686 is then folded down to “tent” tension element 5350.

To remove or exchange anchored device 5100, ball 6110 may be uncoupled from socket 6685.

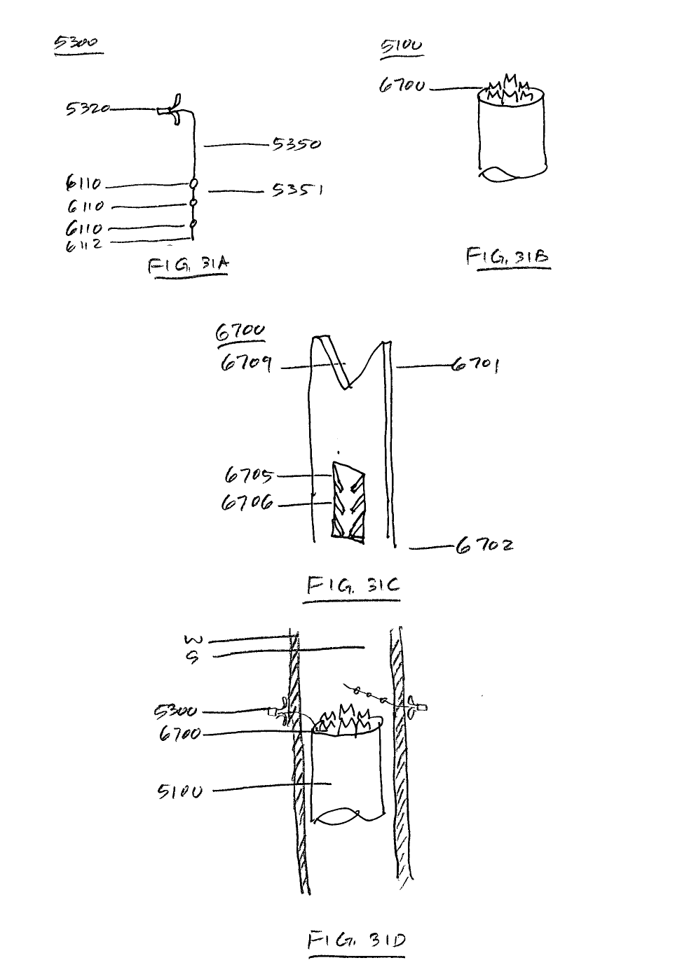

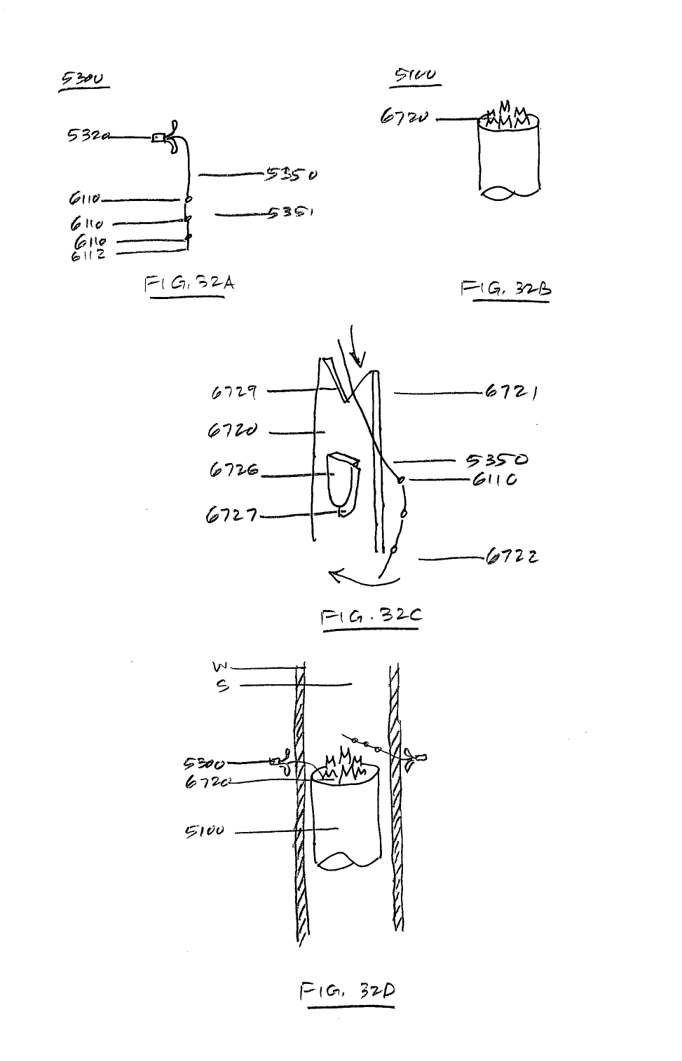

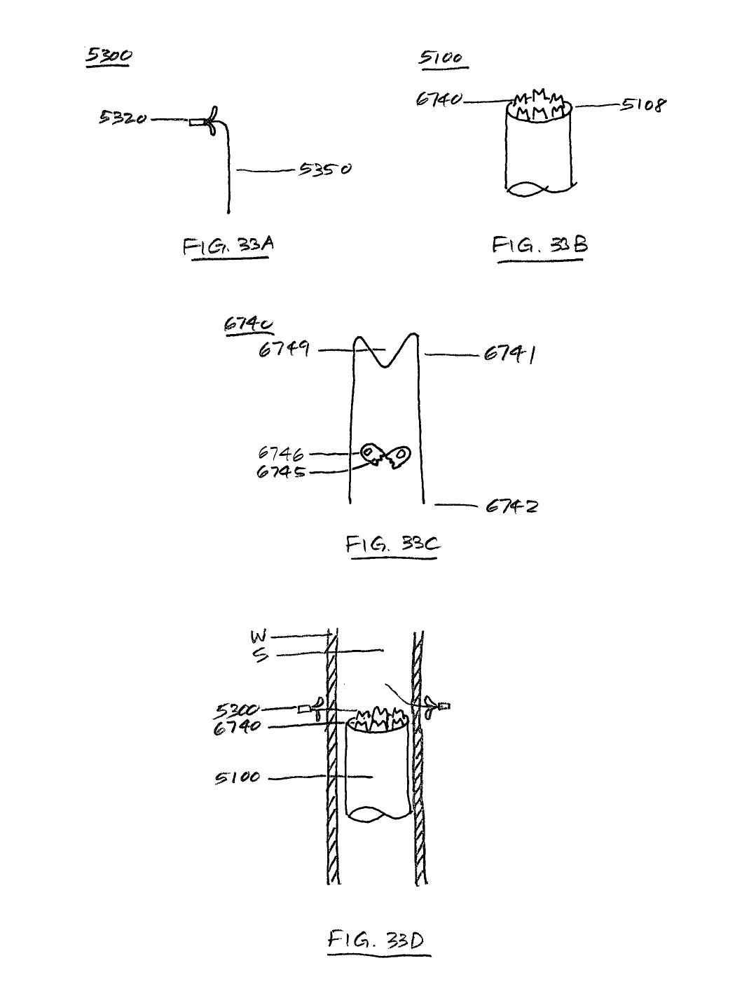

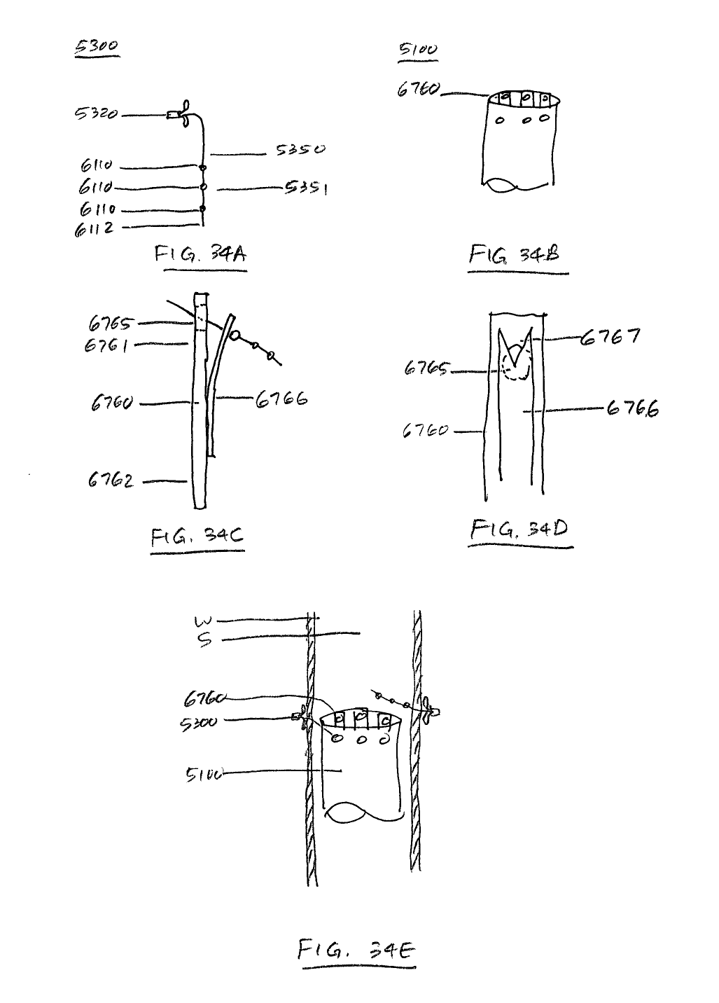

FIG. 31A shows one embodiment of a tissue anchor 5300 including one or more balls 6110. FIG. 31B shows one embodiment of an anchored device 5100 including one or more struts 6700. FIG. 31C shows an enlarged view of one embodiment of strut 6700.

Balls 6110 may include balls or knots along a length of tension element 5350. Balls 6110 may include a pull tab 6112 such as a loop or length of suture.

Strut 6700 includes a proximal portion 6701 and a distal portion 6702. Strut 6700 includes a ratchet 6705 configured to be coupled to ball 6110. Ratchet 6705 includes a plurality of teeth 6706 which allow ball 6110 to be pulled distally but not proximally. Ratchet 6705 is configured to take up at least a portion of any slack in tension element 5350. Strut 6700 may include a notch 6709 at proximal portion 6701. Notch 6709 may be positioned at or near proximal edge 5108 of anchored device 5100. Strut 6700 may be coupled to interior surface 5106 of anchored device 5100.

FIG. 31D shows one embodiment of a method for attaching anchored device 5100 within a bodily space S. One or more tissue anchors 5300 are delivered through the tissue wall W. Anchored device 5100 is delivered into the bodily space S. Ball 6110 is coupled to ratchet 6705. Ball 6110 may be pulled distally to take up a desired amount of slack in tension element 5350. Tension element 5350 is placed in notch 6709.

To remove or exchange anchored device 5100, ball 6110 may be uncoupled from ratchet 6705.

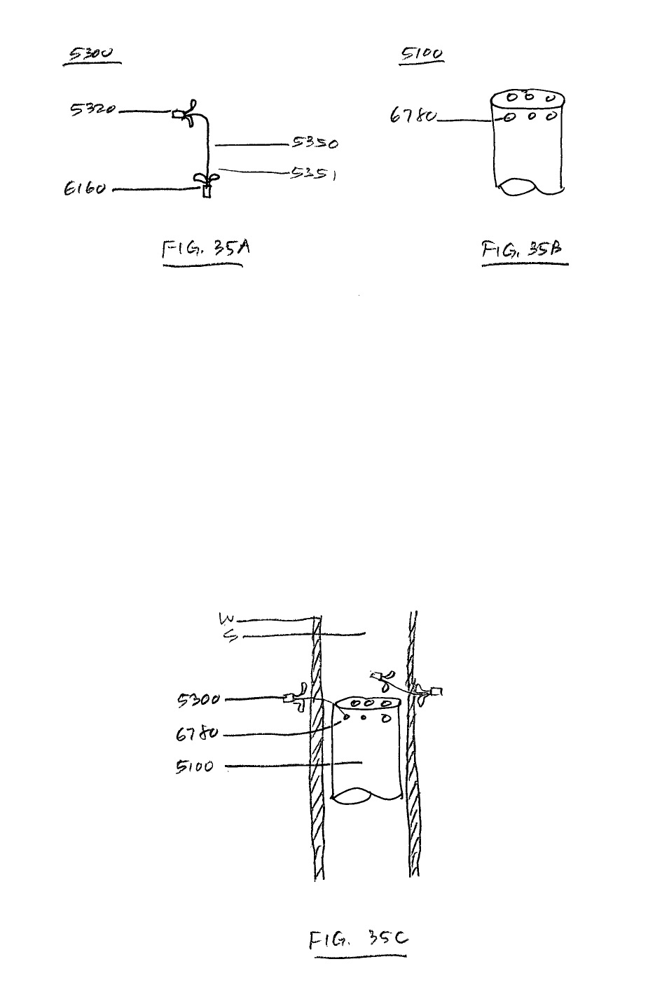

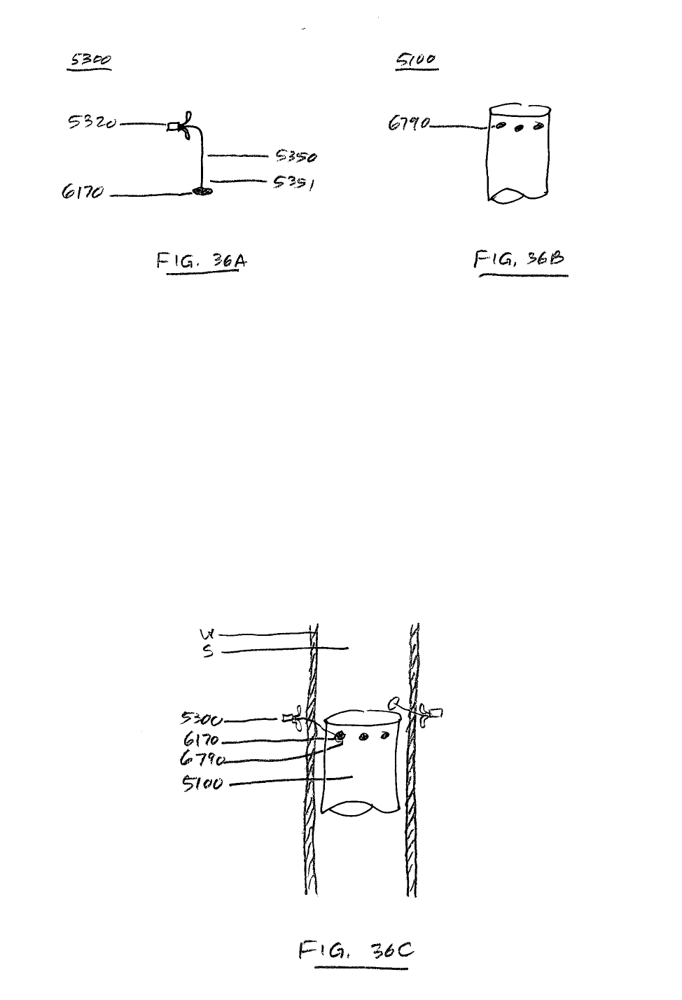

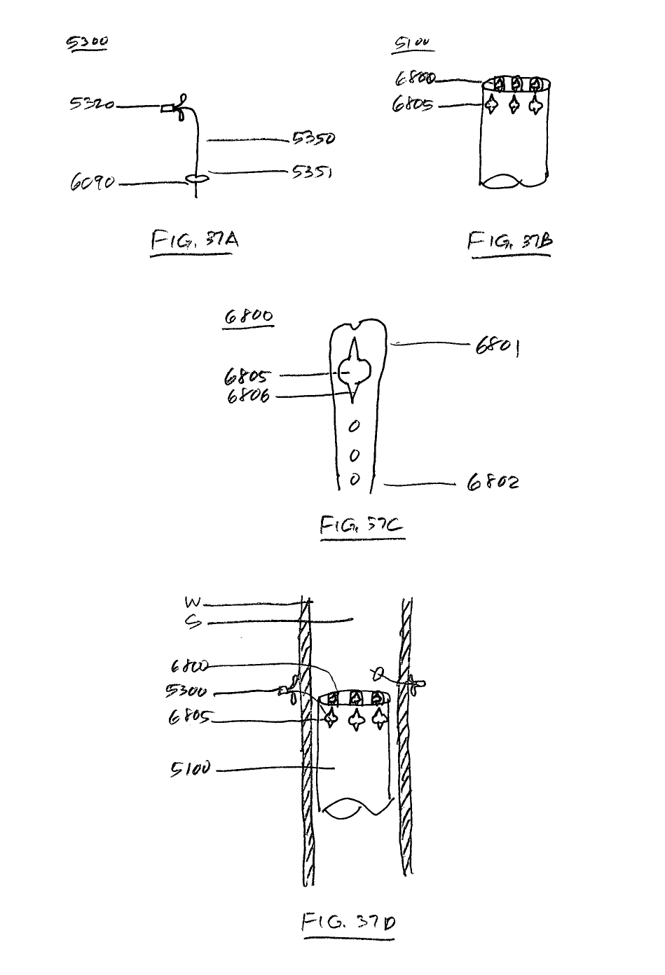

FIG. 32A shows one embodiment of a tissue anchor 5300 including one or more balls 6110. FIG. 32B shows one embodiment of an anchored device 5100 including one or more struts 6720. FIG. 32C shows an enlarged view of one embodiment of strut 6720.

Balls 6110 may include balls or knots along a length of tension element 5350. Balls 6110 may include a pull tab 6112 such as a loop or length of suture.

Strut 6720 includes a proximal portion 6721 and a distal portion 6722. Strut 6720 includes a ratchet 6725 configured to be coupled to ball 6110. Ratchet 6725 includes a spool 6726 with a collar 6727 which allows is configured to be coupled to ball 6110. Ratchet 6725 is configured to take up at least a portion of any slack in tension element 5350. Strut 6720 may include a notch 6729 at proximal portion 6721. Notch 6729 may be positioned at or near proximal edge 5108 of anchored device 5100. Strut 6720 may be coupled to interior surface 5106 of anchored device 5100.

FIG. 32D shows one embodiment of a method for attaching anchored device 5100 within a bodily space S. One or more tissue anchors 5300 are delivered through the tissue wall W. Anchored device 5100 is delivered into the bodily space S. Ball 6110 is coupled to ratchet 6725. Ball 6110 may be pulled around spool 6726 to take up a desired amount of slack in tension element 5350. Tension element 5350 is placed in notch 6729.

To remove or exchange anchored device 5100, ball 6110 may be uncoupled from ratchet 6725.

FIG. 33A shows one embodiment of a tissue anchor 5300. FIG. 33B shows one embodiment of an anchored device 5100 including one or more struts 6740. FIG. 33C shows an enlarged view of one embodiment of strut 6740.

Tissue anchor 5300 may include no anchor coupling 5310 other than tension element 5350.

Strut 6740 includes a proximal portion 6741 and a distal portion 6742. Strut 6740 includes cleats 6745 configured to be coupled to ball 6110. Cleats 6745 may rotate on pivots 6746. Cleats 6745 allow tension element 5350 to travel distally but not proximally. Cleats 6745 are configured to take up at least a portion of any slack in tension element 5350. Strut 6740 may include a notch 6749 at proximal portion 6741. Notch 6749 may be positioned at or near proximal edge 5108 of anchored device 5100. Strut 6740 may be coupled to interior surface 5106 of anchored device 5100.

FIG. 33D shows one embodiment of a method for attaching anchored device 5100 within a bodily space S. One or more tissue anchors 5300 are delivered through the tissue wall W. Anchored device 5100 is delivered into the bodily space S. Tension element 5350 is passed between cleats 6745. Tension element 6745 may be pulled distally to take up a desired amount of slack in tension element 5350. Tension element 5350 is placed in notch 6749.

To remove or exchange anchored device 5100, cleats 6745 may be held open and tension element 5350 removed.

FIG. 34A shows one embodiment of a tissue anchor 5300 including one or more balls 6110. FIG. 34B shows one embodiment of an anchored device 5100 including one or more struts 6760. FIGS. 34C-34D show enlarged views of one embodiment of strut 6760.

Balls 6110 may include balls or knots along a length of tension element 5350. Balls 6110 may include a pull tab 6112 such as a loop or length of suture.

Strut 6760 includes a proximal portion 6761 and a distal portion 6762. Strut 6760 includes a hole 6765 and a retainer 6766 at or near proximal portion 6761. Hole 6765 is large enough for ball 6110 to pass through. Retainer 6766 may include a notch 6767 which at least partially overlaps with hole 6765. Strut 6760 may be coupled to interior surface 5106 of anchored device 5100.

FIG. 34E shows one embodiment of a method for attaching anchored device 5100 within a bodily space S. One or more tissue anchors 5300 are delivered through the tissue wall W. Anchored device 5100 is delivered into the bodily space S. More than one ball 6110 may be passed through hole 6765 to take up a portion of any slack in tension element 5350. Retainer 6766 may flex to allow each ball 6110 to pass through hole 6765. Tension element 5350 is then placed in notch 6767.

To remove or exchange anchored device 5100, retainer 6766 may be held open and balls 6110 removed through hole 6765.

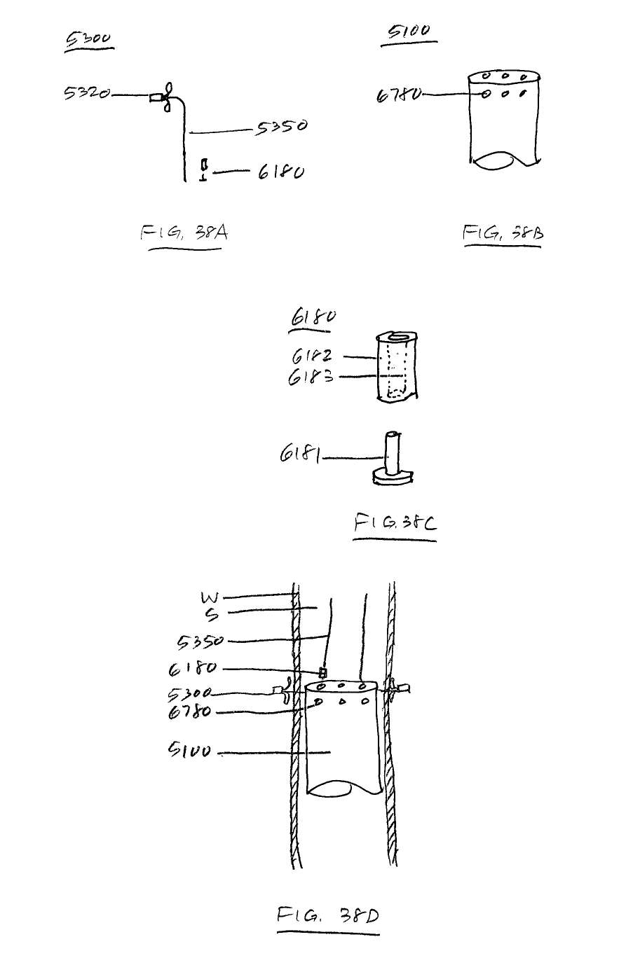

FIG. 35A shows one embodiment of a tissue anchor 5300 including an expanding element 6160. FIG. 35B shows one embodiment of an anchored device 5100 including one or more holes 6780.

Expanding element 6160 is configured to compress while passing through hole 6780 in one direction, but expand after passing completely through hole 6780. Expanding element 6160 may be of a design similar to the distal retention elements described in U.S. patent application Ser. Nos. 12/137,473 and 13/896,838.

FIG. 35C shows one embodiment of a method for attaching anchored device 5100 within a bodily space S. One or more tissue anchors 5300 are delivered through the tissue wall W. Anchored device 5100 is delivered into the bodily space S. Expanding element 6160 is passed completely through hole 6780.

To remove or exchange anchored device 5100, hole 6780 may be cut open.

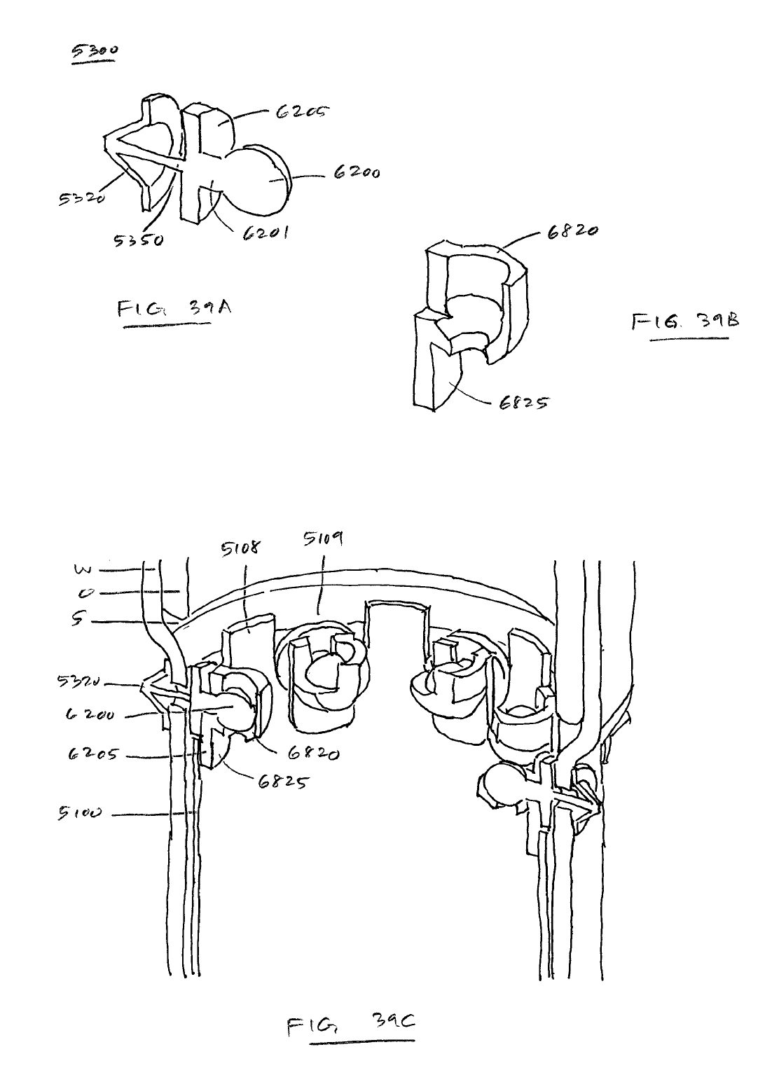

FIG. 36A shows one embodiment of a tissue anchor 5300 including a magnet 6170. FIG. 36B shows one embodiment of an anchored device 5100 including one or more magnets 6790.

Magnet 6170 may be disc-shaped or any other suitable shape.

Magnet 6790 is configured to be coupled to magnet 6170. Magnet 6790 is of a polarity which attracts magnet 6170. Magnet 6790 may be coupled to interior surface 5106, exterior surface 5107, or proximal edge 5108 of anchored device 5100.

FIG. 36C shows one embodiment of a method for attaching anchored device 5100 within a bodily space S. One or more tissue anchors 5300 are delivered through the tissue wall W. Anchored device 5100 is delivered into the bodily space S. Magnet 6170 of tissue anchor 5300 is coupled to magnet 6790 of anchored device 5100.

To remove or exchange anchored device 5100, magnets 6170 are uncoupled from magnets 6790.

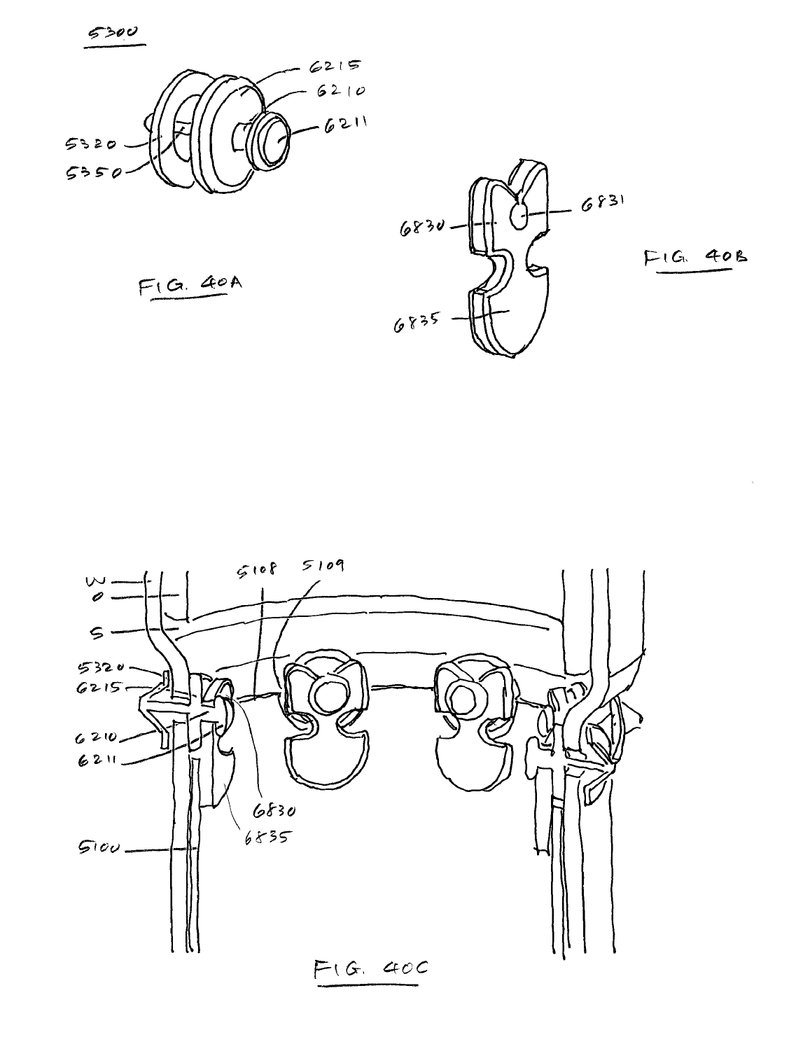

FIG. 37A shows one embodiment of a tissue anchor 5300 including a button 6090. FIG. 37B shows one embodiment of an anchored device 5100 including one or more struts 6800. FIG. 37C shows an enlarged view of one embodiment of strut 6800.

Button 6090 may be round, square, bar-shaped, or any other suitable shape. Button 6090 may include one or more holes 6091 through which proximal portion 5351 of tension element 5350 may be coupled. Button 6090 may allow tension element 5350 to be adjusted in length.

Strut 6800 includes a proximal portion 6801 and a distal portion 6802. Strut 6800 includes a hole 6805 with a slot 6806. Slot 6806 may be oriented substantially longitudinally or in any orientation. Hole 6805 and slot 6806 are configured to allow button 6090 to pass through sideways when it is aligned with slot 6806, and to retain button 6090 once it has passed through completely. Strut 6800 may be coupled to interior surface 5106 or exterior surface 5107 of anchored device 5100.

FIG. 37D shows one embodiment of a method for attaching anchored device 5100 within a bodily space S. One or more tissue anchors 5300 are delivered through the tissue wall W. Anchored device 5100 is delivered into the bodily space S. Button 6090 is passed through hole 6805 and slot 6806.

To remove or exchange anchored device 5100, buttons 6090 may be aligned with slots 6806 and pulled out. Alternatively, holes 6805 may be cut open.

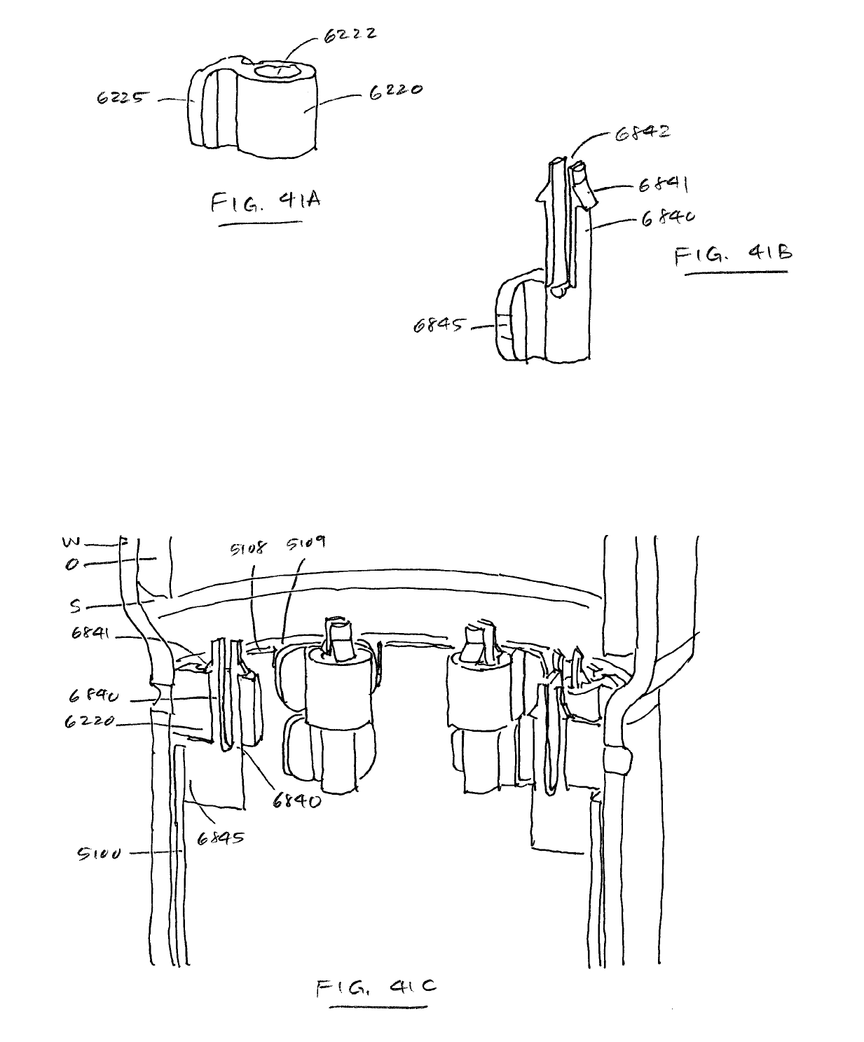

FIG. 38A shows one embodiment of a tissue anchor 5300 including a suture lock 6180. FIG. 38B shows an enlarged view of one embodiment of suture lock 6180. FIG. 38C shows one embodiment of an anchored device 5100 including one or more holes 6780.

Suture lock 6180 includes a pin 6181 and a barrel 6182. Pin 6181 is configured to be inserted into a lumen 6183 of barrel 6182. Pin 6181 may be coupled to barrel 6182 with an interference fit or a snap fit. Pin 6181 and barrel 6182 are configured to be fixedly coupled to tension element 5350 when pin 6181 is coupled to barrel 6182. Tension element 5350 may be long enough to extend out of the bodily space.

FIG. 38D shows one embodiment of a method for attaching anchored device 5100 within a bodily space S. One or more tissue anchors 5300 are delivered through the tissue wall W. Tension element 5350 is passed through hole 6780. Anchored device 5100 may be “parachuted” down over tension element 5350 into the bodily space S. Pin 6181 and barrel 6182 are coupled to tension element 5350 to keep anchored device 5100 in place. Any excess tension element 5350 may then be cut and removed.

To remove or exchange anchored device 5100, pin 6181 and barrel 6182 may be uncoupled from each other and removed from tension element 5350.

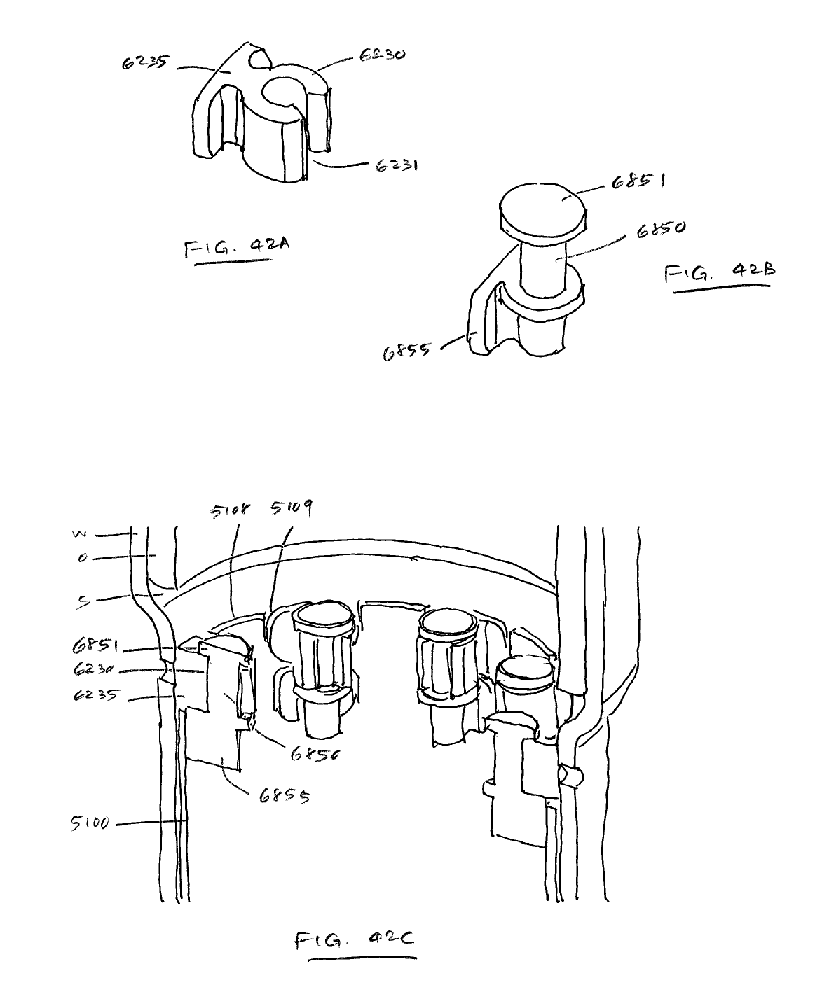

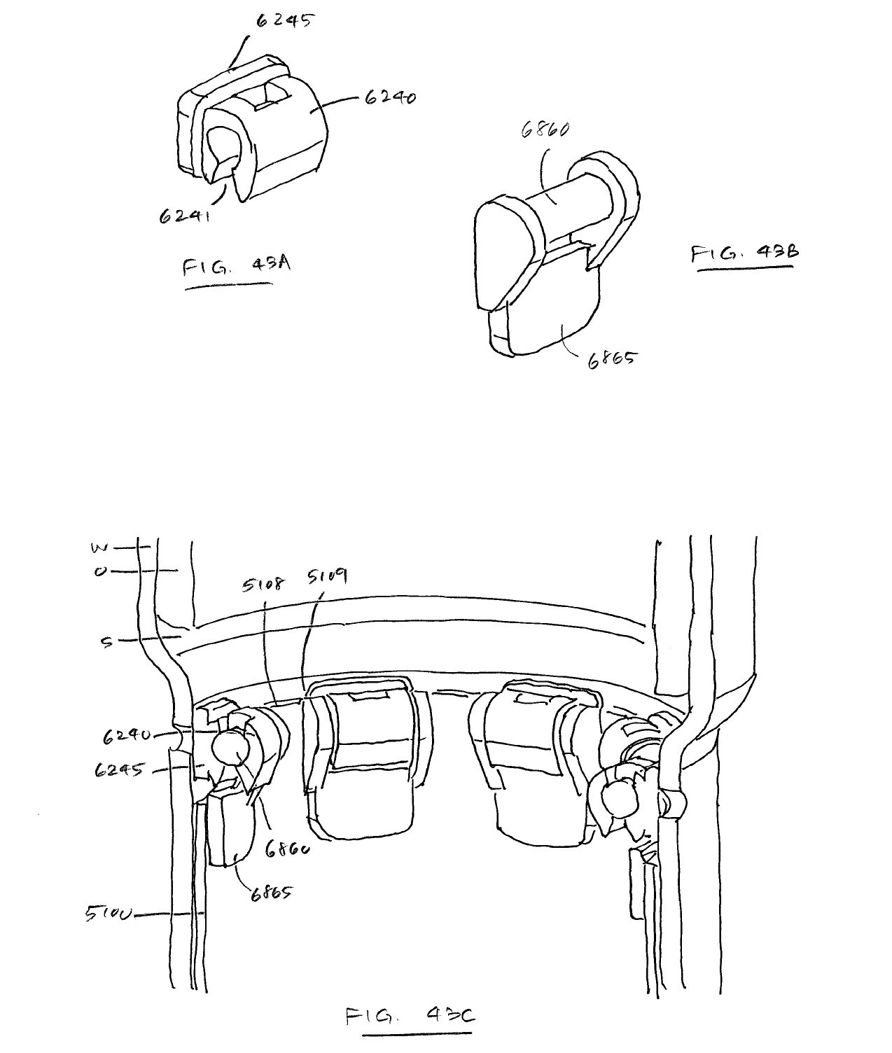

FIG. 39A shows a cross-sectional view of one embodiment of a tissue anchor 5300 including a ball 6200. FIG. 39B shows a cross-sectional view of one embodiment of a socket 6820 of an anchored device 5100.

Ball 6200 may be coupled to a collar 6205 by a stem 6201. Collar 6205 may be configured to be placed near or against a proximal side of the tissue wall. Collar 6205 may be configured to fit in a cutaway 5109 formed at proximal edge 5108 of anchored device 5100.

Socket 6820 may be coupled to ball 6200 with an interference fit or snap fit. Socket 6820 may be coupled to a base 6825 configured to be coupled to anchored device 5100. Socket 6820 may be coupled to proximal edge 5108 of anchored device 5100. Socket 6820 may be positioned at cutaway 5109.

FIG. 39C shows one embodiment of a method for attaching anchored device 5100 within a bodily space S. An overtube O may be used to gain access to the bodily space S. One or more tissue anchors 5300 are delivered through the tissue wall W. Anchored device 5100 is delivered into the bodily space S. Ball 6200 is coupled to socket 6820.

To remove or exchange anchored device 5100, ball 6200 may be uncoupled from socket 6820.

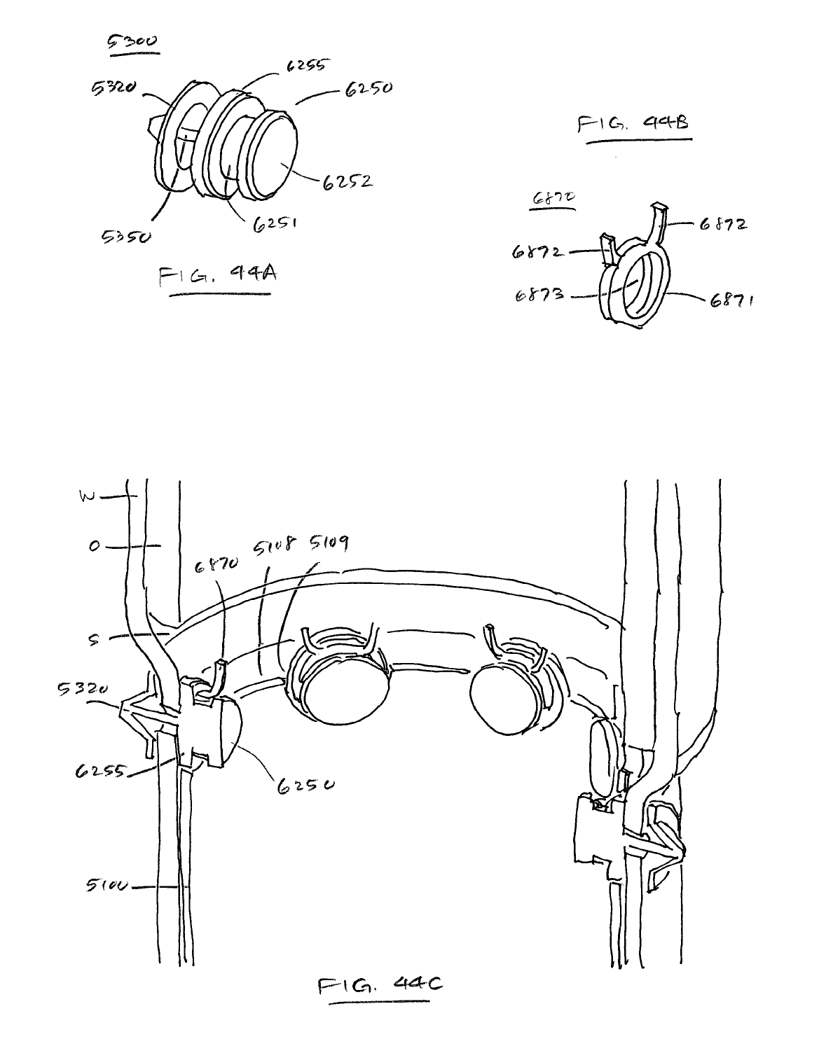

FIG. 40A shows one embodiment of a tissue anchor 5300 including a pin 6210. FIG. 40B shows one embodiment of a clip 6830 of an anchored device 5100.

Pin 6210 may include a retention element 6212 such as a cap. Pin 6210 may be coupled to a collar 6215. Collar 6215 may be configured to be placed near or against a proximal side of the tissue wall. Collar 6215 may be configured to fit in a cutaway 5109 formed at proximal edge 5108 of anchored device 5100.

Clip 6830 may be coupled to pin 6210 with an interference fit or snap fit. Clip 6830 may include a recess 6831 be configured to be coupled to pin 6210. Clip 6830 may be coupled to a base 6835 configured to be coupled to anchored device 5100. Clip 6830 may be coupled to proximal edge 5108 of anchored device 5100. Clip 6830 may be positioned at cutaway 5109.

FIG. 40C shows one embodiment of a method for attaching anchored device 5100 within a bodily space S. An overtube O may be used to gain access to the bodily space S. One or more tissue anchors 5300 are delivered through the tissue wall W. Anchored device 5100 is delivered into the bodily space S. Pin 6210 is coupled to clip 6830.

To remove or exchange anchored device 5100, pin 6210 may be uncoupled from clip 6830.

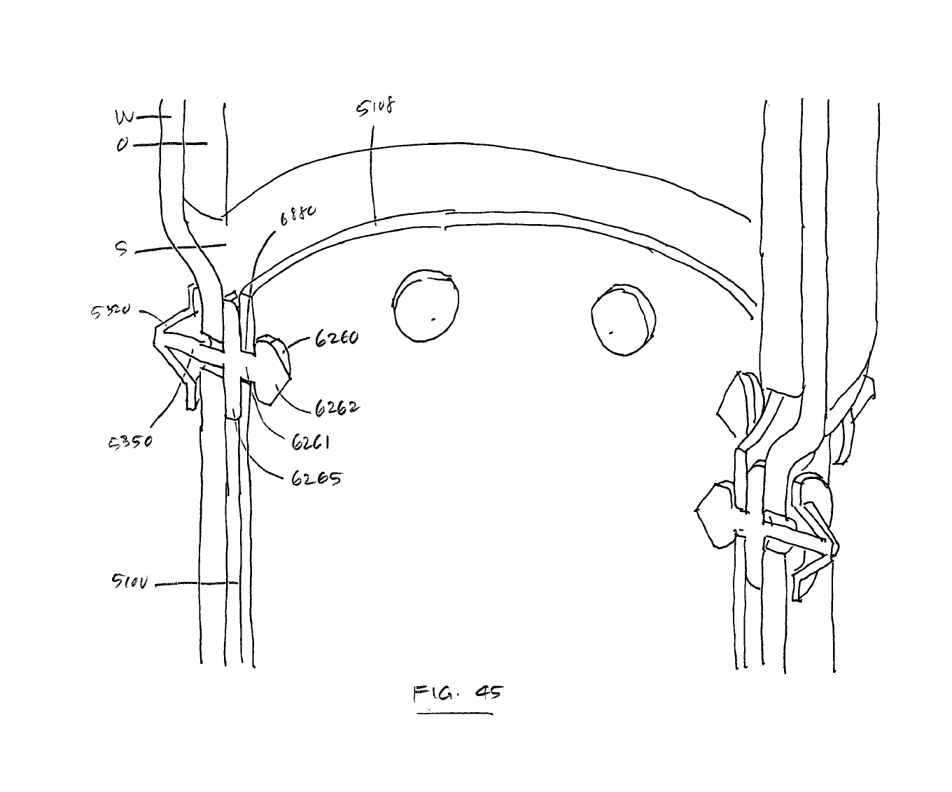

FIG. 41A shows one embodiment of a tube 6220 of a tissue anchor 5300. Distal retention element 5320 and tension element 5350 are not shown for clarity. FIG. 41B shows one embodiment of a pin 6840 of an anchored device 5100.

Tube 6220 may be coupled to a collar 6225. Collar 6225 may be configured to be placed near or against a proximal side of the tissue wall. Collar 6225 may be configured to fit in a cutaway 5109 formed at proximal edge 5108 of anchored device 5100. Tube 6220 may define a lumen 6222.

Pin 6840 may be coupled to tube 6220 with an interference fit or snap fit. Pin 6840 may be inserted into lumen 6222. Pin 6840 may include one or more retention elements 6841 such as barbs. Pin 6840 may include a longitudinal channel 6842 which allows pin 6840 to be compressed. Pin 6840 may be coupled to a base 6845 configured to be coupled to anchored device 5100. Pin 6840 may be coupled to proximal edge 5108 of anchored device 5100. Pin 6840 may be positioned at cutaway 5109.

FIG. 41C shows one embodiment of a method for attaching anchored device 5100 within a bodily space S. An overtube O may be used to gain access to the bodily space S. One or more tissue anchors 5300 are delivered through the tissue wall W. Anchored device 5100 is delivered into the bodily space S. Tube 6220 is coupled to pin 6840.

To remove or exchange anchored device 5100, tube 6220 may be uncoupled from pin 6840.

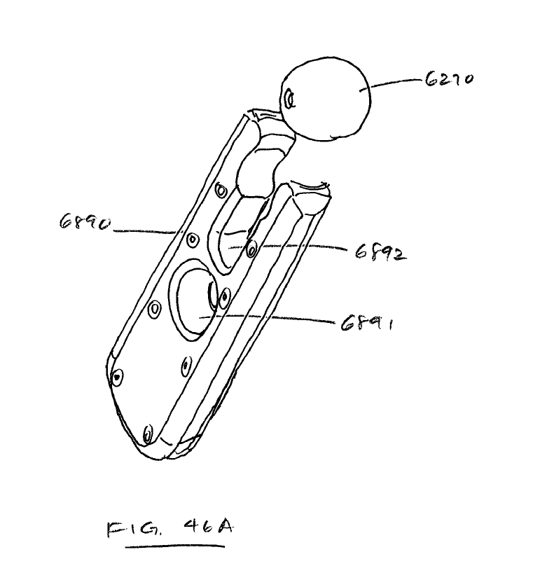

FIG. 42A shows one embodiment of a clip 6230 of a tissue anchor 5300. Distal retention element 5320 and tension element 5350 are not shown for clarity. FIG. 42B shows one embodiment of a pin 6850 of an anchored device 5100.

Clip 6230 may be coupled to a collar 6235. Collar 6235 may be configured to be placed near or against a proximal side of the tissue wall. Collar 6235 may be configured to fit in a cutaway 5109 formed at proximal edge 5108 of anchored device 5100. Clip 6230 may define an opening 6231.

Pin 6850 may be coupled to clip 6230 with an interference fit or snap fit. Pin 6850 may be inserted through opening 6231. Pin 6850 may be coupled to a base 6855 configured to be coupled to anchored device 5100. Pin 6850 may include a retention element 6851 such as a cap. Pin 6850 may be coupled to proximal edge 5108 of anchored device 5100. Pin 6850 may be positioned at cutaway 5109.

FIG. 42C shows one embodiment of a method for attaching anchored device 5100 within a bodily space S. An overtube O may be used to gain access to the bodily space S. One or more tissue anchors 5300 are delivered through the tissue wall W. Anchored device 5100 is delivered into the bodily space S. Clip 6230 is coupled to pin 6850.

To remove or exchange anchored device 5100, clip 6230 may be uncoupled from pin 6850.

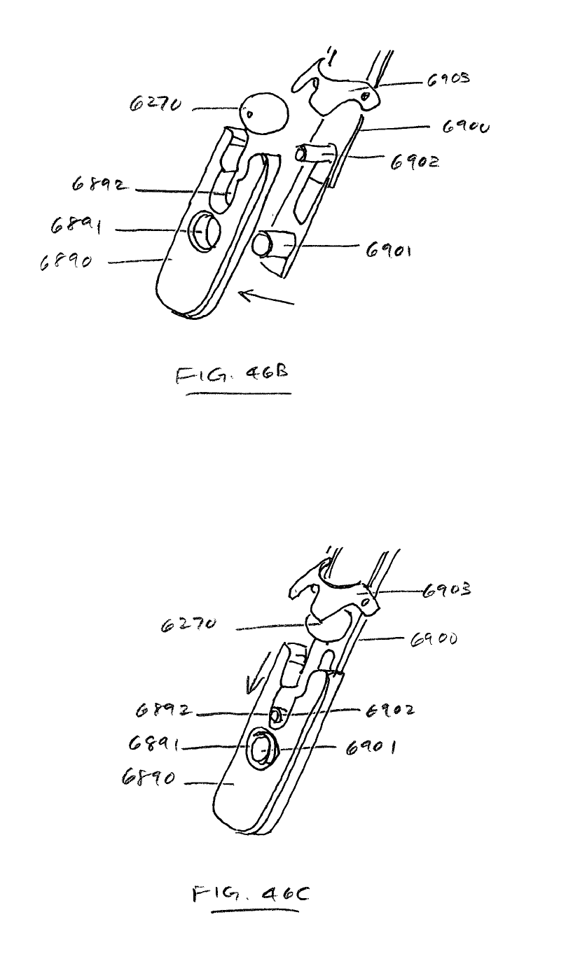

FIG. 43A shows one embodiment of a clip 6240 of a tissue anchor 5300. Distal retention element 5320 and tension element 5350 are not shown for clarity. FIG. 43B shows one embodiment of a pin 6860 of an anchored device 5100.

Clip 6240 may be coupled to a collar 6245. Collar 6245 may be configured to be placed near or against a proximal side of the tissue wall. Collar 6245 may be configured to fit in a cutaway 5109 formed at proximal edge 5108 of anchored device 5100. Clip 6240 may define an opening 6241.

Pin 6860 may be coupled to clip 6240 with an interference fit or snap fit. Pin 6860 may be inserted through opening 6241. Pin 6860 may be coupled at one or both ends to a base 6865 configured to be coupled to anchored device 5100. Pin 6860 may be coupled to proximal edge 5108 of anchored device 5100. Pin 6860 may be positioned at cutaway 5109.

FIG. 43C shows one embodiment of a method for attaching anchored device 5100 within a bodily space S. An overtube O may be used to gain access to the bodily space S. One or more tissue anchors 5300 are delivered through the tissue wall W. Anchored device 5100 is delivered into the bodily space S. Clip 6240 is coupled to pin 6860.

To remove or exchange anchored device 5100, clip 6240 may be uncoupled from pin 6860.

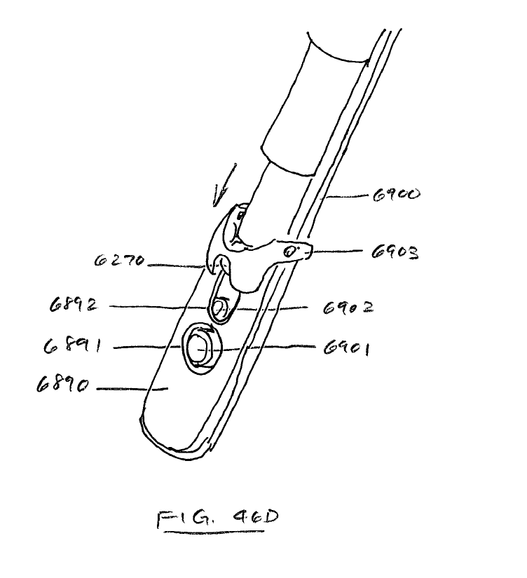

FIG. 44A shows one embodiment of a tissue anchor 5300 including a knob 6250. FIG. 44B shows one embodiment of a clip 6870 of an anchored device 5100.

Knob 6250 may include a stem 6251 and a cap 6252. Knob 6250 may be coupled to a collar 6255. Collar 6255 may be configured to be placed near or against a proximal side of the tissue wall. Collar 6255 may be configured to fit in a cutaway 5109 formed at proximal edge 5108 of anchored device 5100.

Clip 6870 may include one or more windings of a coil spring 6871 with ends 6872. Clip 6870 may be loosened and tightened by squeezing and releasing ends 6872. Clip 6870 defines a hole 6873 which may be large enough for cap 6252 to pass through when clip 6870 is loosened, and may be smaller than cap 6252 when clip 6870 is tightened. Clip 6870 may be coupled to proximal edge 5108 of anchored device 5100. Clip 6870 may be positioned at cutaway 5109.

FIG. 44C shows one embodiment of a method for attaching anchored device 5100 within a bodily space S. An overtube O may be used to gain access to the bodily space S. One or more tissue anchors 5300 are delivered through the tissue wall W. Anchored device 5100 is delivered into the bodily space S. Knob 6250 is coupled to clip 6870.

To remove or exchange anchored device 5100, knob 6250 may be uncoupled from clip 6870.

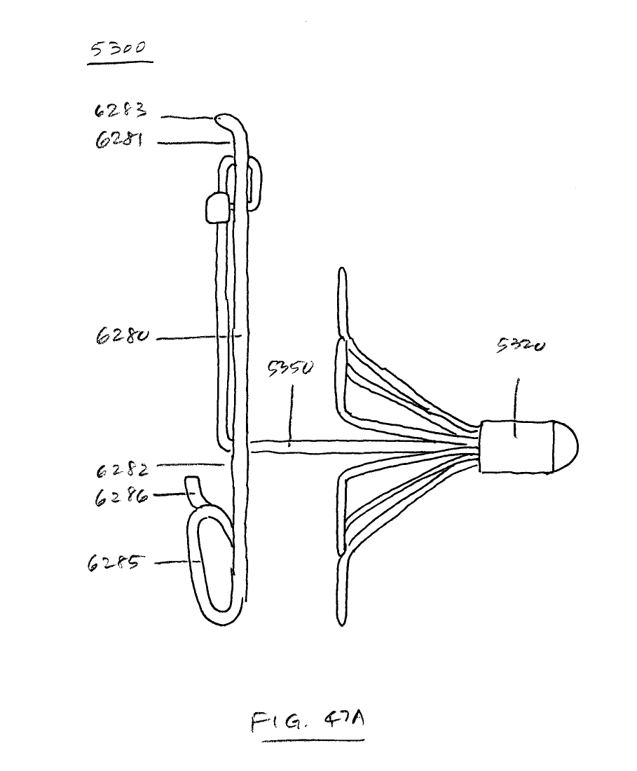

FIG. 45 shows one embodiment of a tissue anchor 5300 including a barb 6260. FIG. 45 also shows one embodiment of an anchored device 5100 including one or more holes 6880.

Barb 6260 may include a stem 6261 and a cap 6262. Cap 6262 may be wider than stem 6261, and may have a generally conical or triangular shape. Barb 6260 may be coupled to a collar 6265. Collar 6265 may be configured to be placed near or against a proximal side of the tissue wall.

FIG. 45 shows one embodiment of a method for attaching anchored device 5100 within a bodily space S. An overtube O may be used to gain access to the bodily space S. One or more tissue anchors 5300 are delivered through the tissue wall W. Anchored device 5100 is delivered into the bodily space S. Barb 6260 is coupled to hole 6880.

To remove or exchange anchored device 5100, barb 6260 may be uncoupled from hole 6880.

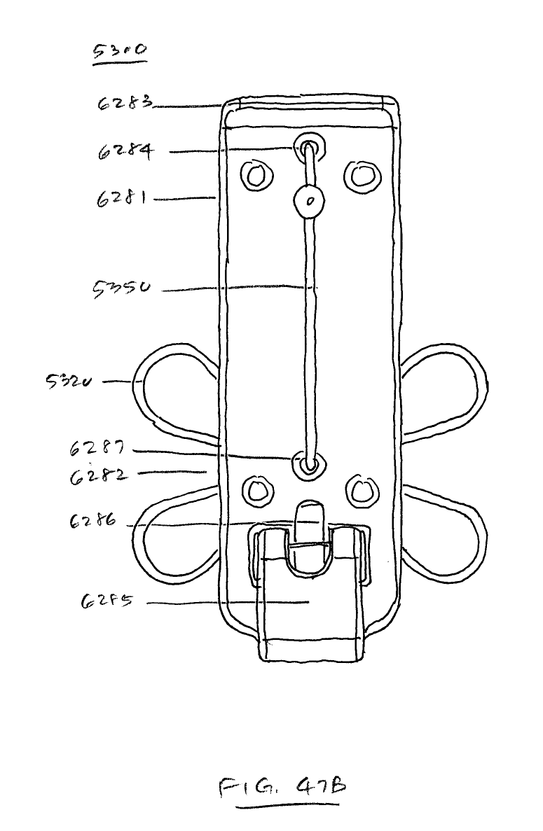

FIG. 46A shows one embodiment of a ball 6270 of a tissue anchor 5300. Distal retention element 5320 and tension element 5350 are not shown for clarity. FIG. 46A also shows one embodiment of a socket 6890 of an anchored device 5100.

Socket 6890 may be coupled to ball 6270 with an interference fit or snap fit. Socket 6890 may include a tool features 6891 and 6892 which may include holes, protrusions, and/or recesses. Socket 6890 may be coupled at or near proximal edge 5108 of anchored device 5100. Socket 6890 may be coupled to interior surface 5106 or exterior surface 5107 of anchored device 5100.

FIGS. 46B-46D show one embodiment of a method for coupling ball 6270 to socket 6890. A coupling tool 6900 includes coupling elements 6901 and 6902. Coupling element 6901 and 6902 configured to grasp socket 6890 through tool features 6891 and 6892. In the embodiment shown, coupling element 6901 and 6902, which include independently movable posts, fit into tool features 6891 and 6892, which include holes and/or recesses. Coupling tool 6900 also includes a hood 6903 configured to fit over and hold ball 6270.

FIG. 46B shows aligning coupling tool 6900 with ball 6270 and socket 6890.

FIG. 46C shows inserting coupling elements 6901 and 6902 through tool features 6891 and 6892 and slidably adjusting coupling elements 6901 and 6902 to grasp socket 6890. Hood 6903 is placed over ball 6270.

FIG. 46D shows using hood 6903 to push ball 6270 into socket 6890. When pressed against socket 6890, hood 6903 may be configured to rotate and hold ball 6270 more securely.

To remove or exchange anchored device 5100, ball 6270 may be uncoupled from socket 6890.

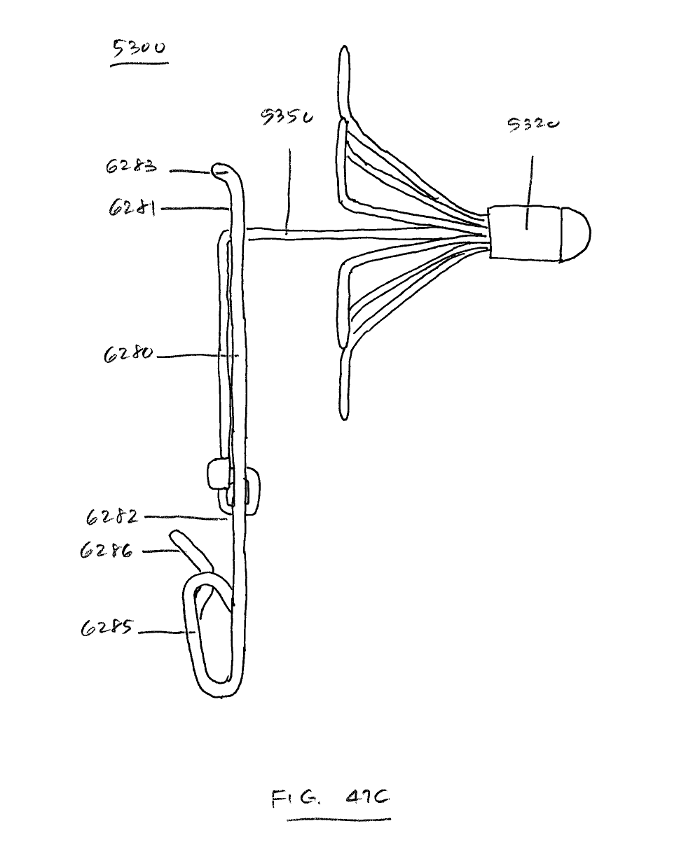

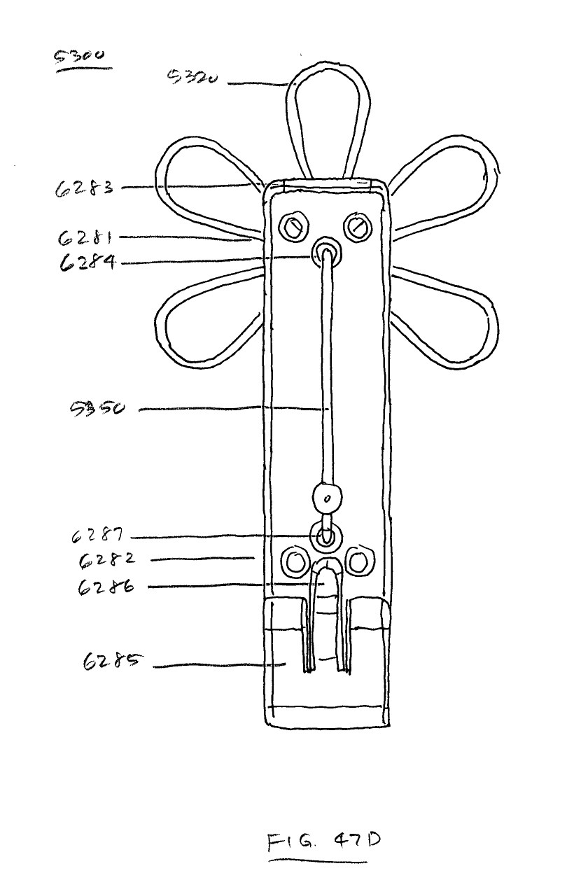

FIGS. 47A-47B show side and front views of one embodiment of a tissue anchor 5300 including a folding member 6280. FIGS. 47C-47D show side and front views of another embodiment of a tissue anchor 5300 including a folding member 6280.

Folding member 6280 includes a proximal portion 6281 and a distal portion 6282. Proximal portion 6281 may include a lip 6283. Proximal portion 6281 may also include a proximal hole 6284. Distal portion 6282 may include a hook 6285 with a tang 6286. Distal portion 6282 may also include a distal hole 6287. Tension element 5350 may pass through either proximal hole 6284 or distal hole 6287 and be secured at distal portion 6282 or proximal portion 6281, respectively.

Folding member 6280 is biased in a substantially straight configuration. Folding member 6280 may be folded so that lip 6283 is retained in tang 6286. This produces slack in tension element 5350, which may facilitate delivery of distal retention element 5320 through a tissue wall.

After distal retention element 5320 is delivered through a tissue wall, tang 6286 may be pulled to release lip 6283 and allow folding member 6280 to return to a substantially straight configuration. This takes up slack in tension element 5350.

Hook 6285 is configured to be coupled to anchored device 5100 having a hole, loop, or any other suitable device or structure.

While the foregoing has been with reference to particular embodiments of the invention, it will be appreciated by those skilled in the art that changes in these embodiments may be made without departing from the principles and spirit of the invention, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or subcombinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described above. For all of the embodiments described above, the steps of any methods need not be performed sequentially.

1. A method of treating a patient for a condition, the method comprising:
providing a tissue anchor including a tension element, an anchor coupling coupled to a proximal portion of the tension element, and a distal retention element coupled to a distal portion of the tension element, the anchor coupling configured to be positioned within a bodily space, the distal retention element configured to be deployed on a distal side of a tissue wall defining the bodily space;
delivering the tissue anchor into the bodily space;
delivering the distal retention element through the tissue wall;
deploying the distal retention element on the distal side of the tissue wall;
positioning the anchor coupling within the bodily space;
providing an anchored device configured to be positioned within the bodily space, the anchored device including a device coupling configured to be coupled to the anchor coupling;
delivering the anchored device into the bodily space; and
coupling the device coupling to the anchor coupling to anchor the anchored device in the bodily space.

2. The method of claim 1, wherein the anchor coupling includes a loop, barb, hook, ball and socket, fork, clip, halo, cinching loop, button, T-tag, ball, knob, insert, pop cap, pincer, expanding device, magnet, suture lock, ball, pin, tube, and/or folding member.

3. The method of claim 1, wherein the device coupling includes a clip, barb, loop, hitch, strut, fin, loop coupling, halo, knob, cinching loop, ring, block, cleats, hole, magnet, socket, and/or pin.

4. The method of claim 1, wherein the bodily space includes an esophageal lumen.

5. The method of claim 1, wherein the tissue wall includes an esophageal wall.

6. The method of claim 1, wherein the anchored device includes a gastrointestinal cuff, gastrointestinal sleeve, gastrointestinal bypass device, and/or GERD device.

7. The method of claim 1, wherein coupling the device coupling to the anchor coupling includes removably coupling the device coupling to the anchor coupling.

8. The method of claim 1, further comprising:
uncoupling the device coupling from the anchor coupling;
removing the anchored device from the bodily space;
providing a new anchored device configured to be positioned within the bodily space, the new anchored device including a new device coupling configured to be coupled to the anchor coupling;
delivering the new anchored device into the bodily space;
coupling the new device coupling to the anchor coupling to anchor the new anchored device in the bodily space.

9. The method of claim 8, wherein coupling the new device coupling to the anchor coupling includes removably coupling the new device coupling to the anchor coupling.

10. A method of treating a patient for a condition, the method comprising:
providing a tissue anchor including a tension element, an anchor coupling coupled to a proximal portion of the tension element, and a distal retention element coupled to a distal portion of the tension element, the anchor coupling configured to be positioned within a bodily space, the distal retention element configured to be deployed on a distal side of a tissue wall defining the bodily space;
selecting a placement in the tissue wall for the tissue anchor;
delivering the distal retention element through the placement in the tissue wall;
deploying the distal retention element on the distal side of the tissue wall;
positioning the anchor coupling within the bodily space;
providing an anchored device configured to be positioned within the bodily space, the anchored device including a device coupling configured to be coupled to the anchor coupling;
delivering the anchored device into the bodily space; and
coupling the device coupling to the anchor coupling to anchor the anchored device in the bodily space.

11. The method of claim 10, wherein the anchor coupling includes a loop, barb, hook, ball and socket, fork, clip, halo, cinching loop, button, T-tag, ball, knob, insert, pop cap, pincer, expanding device, magnet, suture lock, ball, pin, tube, and/or folding member.

12. The method of claim 10, wherein the device coupling includes a clip, barb, loop, hitch, strut, fin, loop coupling, halo, knob, cinching loop, ring, block, cleats, hole, magnet, socket, and/or pin.

13. The method of claim 10, wherein the bodily space includes an esophageal lumen.

14. The method of claim 10, wherein the tissue wall includes an esophageal wall.

15. The method of claim 10, wherein the anchored device includes a gastrointestinal cuff, gastrointestinal sleeve, gastrointestinal bypass device, and/or GERD device.

16. The method of claim 10, wherein coupling the device coupling to the anchor coupling includes removably coupling the device coupling to the anchor coupling.

17. The method of claim 10, further comprising:
uncoupling the device coupling from the anchor coupling;
removing the anchored device from the bodily space;
providing a new anchored device configured to be positioned within the bodily space, the new anchored device including a new device coupling configured to be coupled to the anchor coupling;
delivering the new anchored device into the bodily space;
coupling the new device coupling to the anchor coupling to anchor the new anchored device in the bodily space.

18. The method of claim 17, wherein coupling the new device coupling to the anchor coupling includes removably coupling the new device coupling to the anchor coupling.

19-22. (canceled)
